(19) United States
(12) Patent Application Publication
(10) Pub. No.: US 2023/0073692 A1
(43) Pub. Date: Mar. 9, 2023

(54) ANTI-SLC34A2 ANTIBODIES, ANTIBODY DRUG CONJUGATES, AND METHODS OF USE THEREOF

(71) Applicant: CYTOMX THERAPEUTICS, INC., South San Francisco, CA (US)

(72) Inventors: Dowdy Jackson, South San Francisco, CA (US); Yuriy Shostak, South San Francisco, CA (US); Christopher Kemball, South San Francisco, CA (US); Pia Challita-Eid, South San Francisco, CA (US)

(21) Appl. No.: 17/790,838

(22) PCT Filed: Jan. 6, 2021
(86) PCT No.: PCT/US2021/012383

§ 371 Date: Jul. 5, 2022

Related U.S. Application Data
(60) Provisional application No. 62/957,780, filed on Jan. 6, 2020.

(51) Int. Cl. C07K 16/28; A61K 47/68; A61K 39/395; A61P 35/00; A61P 15/00; A61P 35/04; A61P 11/00

(52) U.S. Cl. CPC C07K 16/28; A61K 47/6849; A61K 47/6817; A61K 39/3955; A61P 35/00; A61P 15/00; A61P 35/04; A61P 11/00; A61K 2039/505

(57) ABSTRACT

The invention relates generally to antibodies that bind SLC34A2, and methods of making and using these anti-SLC34A2 antibodies in a variety of therapeutic, diagnostic and prophylactic indications.

### RELATED APPLICATIONS

This application claims the benefit of U.S. Provisional Application Nos. 62/957,780, filed Jan. 6, 2020, the contents of which are incorporated herein by reference in its entirety.

### REFERENCE TO SEQUENCE LISTING

The Sequence Listing submitted electronically concurrently herewith pursuant 37 C.F.R. § 1.821 in computer readable form (ASCII format) via EFS-Web as file name CYTX_078_PCT_ST25.txt is incorporated herein by reference. The ASCII copy of the Sequence Listing was created on Jan. 6, 2021 and is 48 kilobytes in size.

### FIELD OF THE INVENTION

The invention relates generally to antibodies that bind SLC34A2, and using these anti-SLC34A2 antibodies in a variety of therapeutic, diagnostic and prophylactic indications.

### BACKGROUND OF THE INVENTION

Antibody-based therapies have proven effective treatments for several diseases. In some cases, antibodies have found additional usefulness by conjugating them to agents, such as cytotoxic compounds. Such conjugated antibodies, also known as antibody drug conjugates (ADCs) allow the target-specific delivery of the conjugated toxin to cells or tissues that express the target of the antibody. In this manner, the ADC provides a way to specifically deliver a cytotoxic compound based on the antibody specificity.

Accordingly, there is a continued need in the field of new antibodies and ADCs to new molecular targets.

### SUMMARY OF THE INVENTION

The disclosure provides antibodies or antigen-binding fragments thereof that specifically bind SLC34A2, also known as. The use of the term “SLC34A2” is intended to cover any variation thereof, and all variations are used herein interchangeably.

In some embodiments, the antibody includes an antibody or antigen-binding fragment thereof that specifically binds SLC34A2. In some embodiments, the antibody or antigen-binding fragment thereof that binds SLC34A2 is a monoclonal antibody, domain antibody, single chain, Fab fragment, a F(ab′)₂ fragment, a scFv, a scAb, a dAb, a single domain heavy chain antibody, or a single domain light chain antibody. In some embodiments, such an antibody or antigen-binding fragment thereof that binds SLC34A2 is a mouse, other rodent, chimeric, humanized or fully human monoclonal antibody.

In some embodiments, the antibody or antigen-binding fragment thereof comprises a heavy chain variable region amino acid sequence comprising SEQ ID NO: 67 or SEQ ID NO: 68. In some embodiments, the antibody or antigen-binding fragment thereof comprises a heavy chain variable region amino acid sequence comprising SEQ ID NO: 67. In some embodiments, the antibody or antigen-binding fragment thereof comprises a heavy chain variable region amino acid sequence comprising SEQ ID NO: 68.

In some embodiments, the antibody or antigen-binding fragment thereof comprises a light chain variable region amino acid sequence comprising SEQ ID NO: 67 or SEQ ID NO: 68. In some embodiments, the antibody or antigen-binding fragment thereof comprises a light chain variable region amino acid sequence comprising SEQ ID NO: 67. In some embodiments, the antibody or antigen-binding fragment thereof comprises a light chain variable region amino acid sequence comprising SEQ ID NO: 68.

In some embodiments, the antibody or antigen-binding fragment thereof comprises a heavy chain variable region amino acid sequence selected from the group consisting of SEQ ID NO: 67 and SEQ ID NO: 68, and a light chain variable region amino acid sequence selected from the group consisting of SEQ ID NO: 67 and SEQ ID NO: 68. In some embodiments, the antibody or antigen-binding fragment thereof comprises a heavy chain variable region amino acid sequence of SEQ ID NO: 67, and a light chain variable region amino acid sequence of SEQ ID NO: 67. In some embodiments, the antibody or antigen-binding fragment thereof comprises a heavy chain variable region amino acid sequence of SEQ ID NO: 68, and a light chain variable region amino acid sequence of SEQ ID NO: 68.

In some embodiments, the antibody or antigen-binding fragment thereof comprises a heavy chain variable region amino acid sequence that is at least 90%, 91%, 92%, 93%, 94%, 95%, 96%, 97%, 98% or 99% identical to an amino acid sequence comprising SEQ ID NO: 67 or SEQ ID NO: 68. In some embodiments, the antibody or antigen-binding fragment thereof comprises a heavy chain variable region amino acid sequence that is at least 90%, 91%, 92%, 93%, 94%, 95%, 96%, 97%, 98% or 99% identical to an amino acid sequence comprising SEQ ID NO: 68. In some embodiments, the antibody or antigen-binding fragment thereof comprises a heavy chain variable region amino acid sequence that is at least 90%, 91%, 92%, 93%, 94%, 95%, 96%, 97%, 98% or 99% identical to an amino acid sequence comprising SEQ ID NO: 67.

In some embodiments, the antibody or antigen-binding fragment thereof comprises a light chain variable region amino acid sequence that is at least 90%, 91%, 92%, 93%, 94%, 95%, 96%, 97%, 98% or 99% identical to an amino acid sequence comprising SEQ ID NO: 67 or SEQ ID NO: 68. In some embodiments, the antibody or antigen-binding fragment thereof comprises a light chain variable region amino acid sequence that is at least 90%, 91%, 92%, 93%, 94%, 95%, 96%, 97%, 98% or 99% identical to an amino acid sequence comprising SEQ ID NO: 67. In some embodiments, the antibody or antigen-binding fragment thereof comprises a light chain variable region amino acid sequence that is at least 90%, 91%, 92%, 93%, 94%, 95%, 96%, 97%, 98% or 99% identical to the amino acid sequence SEQ ID NO: 68.

In some embodiments, the antibody or antigen-binding fragment thereof comprises a heavy chain variable region amino acid sequence that is at least 90%, 91%, 92%, 93%, 94%, 95%, 96%, 97%, 98% or 99% identical to an amino acid sequence selected from the group comprising SEQ ID NO: 67 or SEQ ID NO: 68, and a light chain variable region amino acid sequence that is at least 90%, 91%, 92%, 93%, 94%, 95%, 96%, 97%, 98% or 99% identical to an amino acid sequence selected from the group comprising SEQ ID NO: 67 or SEQ ID NO: 68.

In some embodiments, the antibody or antigen-binding fragment thereof comprises a heavy chain variable region amino acid sequence that is at least 90%, 91%, 92%, 93%, 94%, 95%, 96%, 97%, 98% or 99% identical to an amino acid sequence SEQ ID NO: 67, and a light chain variable region amino acid sequence that is at least 90%, 91%, 92%, 93%, 94%, 95%, 96%, 97%, 98% or 99% identical to an amino acid sequence comprising SEQ ID NO: 67.

In some embodiments, the antibody or antigen-binding fragment thereof comprises a heavy chain variable region amino acid sequence that is at least 90%, 91%, 92%, 93%, 94%, 95%, 96%, 97%, 98% or 99% identical to an amino acid sequence comprising SEQ ID NO: 68, and a light chain variable region amino acid sequence that is at least 90%, 91%, 92%, 93%, 94%, 95%, 96%, 97%, 98% or 99% identical to an amino acid sequence comprising SEQ ID NO: 68.

In some embodiments, the antibody or antigen-binding fragment thereof comprises a combination of a variable heavy chain complementarity determining region 1 (VH CDR1, also referred to herein as CDRH1) sequence, a variable heavy chain complementarity determining region 2 (VH CDR2, also referred to herein as CDRH2) sequence, a variable heavy chain complementarity determining region 3 (VH CDR3, also referred to herein as CDRH3) sequence, a variable light chain complementarity determining region 1 (VL CDR1, also referred to herein as CDRL1) sequence, a variable light chain complementarity determining region 2 (VL CDR2, also referred to herein as CDRL2) sequence, and a variable light chain complementarity determining region 3 (VL CDR3, also referred to herein as CDRL3) sequence, wherein at least one complementarity determining region (CDR) sequence is selected from the group consisting of a VH CDR1 sequence comprising the amino acid sequence SYVMH (SEQ ID NO: 43) or SHIMY (SEQ ID NO: 46); a VH CDR2 sequence comprising the amino acid sequence GVSSSGDSTFYVDSVKG (SEQ ID NO: 44) or GISSNGLSSYYVDSVKG (SEQ ID NO: 47); a VH CDR3 sequence comprising the amino acid sequence GGITGAPLVFDI (SEQ ID NO: 45) or GGRDRVPAVFDY (SEQ ID NO: 48); a VL CDR1 sequence comprising the amino acid sequence RASQSISRFLN (SEQ ID NO: 37) or RASQSIGTFLN (SEQ ID NO: 40); a VL CDR2 sequence comprising the amino acid sequence VTSSLQS (SEQ ID NO: 38) or VASSLQS (SEQ ID NO: 41); and a VL CDR3 sequence comprising the amino acid sequence QQSYNTPIT (SEQ ID NO: 39) or QQSYSVPIT (SEQ ID NO: 42).

In some embodiments, the antibody or antigen-binding fragment thereof comprises a combination of a VH CDR1 sequence, a VH CDR2 sequence, a VH CDR3 sequence, a VL CDR1 sequence, a VL CDR2 sequence, and a VL CDR3 sequence, wherein at one complementarity determining region (CDR) sequence is selected from the group consisting of a VH CDR1 sequence comprising the amino acid sequence SYVMH (SEQ ID NO: 43); a VH CDR2 sequence comprising the amino acid sequence GVSSSGDSTFYVDSVKG (SEQ ID NO: 44); a VH CDR3 sequence comprising the amino acid sequence GGITGAPLVFDI (SEQ ID NO: 45), a VL CDR1 sequence comprising the amino acid sequence RASQSISRFLN (SEQ ID NO: 37); a VL CDR2 sequence comprising the amino acid sequence VTSSLQS (SEQ ID NO: 38); and a VL CDR3 sequence comprising the amino acid sequence QQSYNTPIT (SEQ ID NO: 39).

In some embodiments, the antibody or antigen-binding fragment thereof comprises a combination of a VH CDR1 sequence, a VH CDR2 sequence, a VH CDR3 sequence, a VL CDR1 sequence, a VL CDR2 sequence, and a VL CDR3 sequence, wherein at least one CDR sequence is selected from the group consisting of a VH CDR1 sequence that includes a sequence that is at least 90%, 91%, 92%, 93%, 94%, 95%, 96%, 97%, 98%, 99% or more identical to a VH CDR1 sequence comprising the amino acid sequence SYVMH (SEQ ID NO: 43); a VH CDR2 sequence that includes a sequence that is at least 90%, 91%, 92%, 93%, 94%, 95%, 96%, 97%, 98%, 99% or more identical to a VH CDR2 sequence comprising the amino acid sequence GVSSSGDSTFYVDSVKG (SEQ ID NO: 44); a VH CDR3 sequence that includes a sequence that is at least 90%, 91%, 92%, 93%, 94%, 95%, 96%, 97%, 98%, 99% or more identical to a VH CDR3 sequence comprising the amino acid sequence GGITGAPLVFDI (SEQ ID NO: 45); a VL CDR1 sequence that includes a sequence that is at least 90%, 91%, 92%, 93%, 94%, 95%, 96%, 97%, 98%, 99% or more identical to a VL CDR1 sequence comprising the amino acid sequence comprising the amino acid sequence RASQSISRFLN (SEQ ID NO: 37); a VL CDR2 sequence that includes a sequence that is at least 90%, 91%, 92%, 93%, 94%, 95%, 96%, 97%, 98%, 99% or more identical to a VL CDR2 sequence comprising the amino acid sequence VTSSLQS (SEQ ID NO: 38); and a VL CDR3 sequence that includes a sequence that is at least 90%, 91%, 92%, 93%, 94%, 95%, 96%, 97%, 98%, 99% or more identical to a VL CDR3 sequence comprising the amino acid sequence QQSYNTPIT (SEQ ID NO: 39).

In some embodiments, the antibody or antigen-binding fragment thereof comprises a combination of a VH CDR1 sequence, a VH CDR2 sequence, a VH CDR3 sequence, a VL CDR1 sequence, a VL CDR2 sequence, and a VL CDR3 sequence, wherein at least one complementarity determining region (CDR) sequence is selected from the group consisting of a VH CDR1 sequence comprising the amino acid sequence SHIMY (SEQ ID NO: 46); a VH CDR2 sequence comprising the amino acid sequence GISSNGLSSYYVDSVKG (SEQ ID NO: 47); a VH CDR3 sequence comprising the amino acid sequence GGRDRVPAVFDY (SEQ ID NO: 48); a VL CDR1 sequence comprising the amino acid sequence RASQSIGTFLN (SEQ ID NO: 40); a VL CDR2 sequence comprising the amino acid sequence VASSLQS (SEQ ID NO: 41), and a VL CDR3 sequence comprising the amino acid sequence QQSYSVPIT (SEQ ID NO: 42).

In some embodiments, the antibody or antigen-binding fragment thereof comprises a combination of a VH CDR1 sequence, a VH CDR2 sequence, a VH CDR3 sequence, a VL CDR1 sequence, a VL CDR2 sequence, and a VL CDR3 sequence, wherein at least one CDR sequence is selected from the group consisting of a VH CDR1 sequence that includes a sequence that is at least 90%, 91%, 92%, 93%, 94%, 95%, 96%, 97%, 98%, 99% or more identical to a VH CDR1 sequence comprising the amino acid sequence SHIMY (SEQ ID NO: 46); a VH CDR2 sequence that includes a sequence that is at least 90%, 91%, 92%, 93%, 94%, 95%, 96%, 97%, 98%, 99% or more identical to a VH CDR2 sequence comprising the amino acid sequence GISSNGLSSYYVDSVKG (SEQ ID NO: 47); a VH CDR3 sequence that includes a sequence that is at least 90%, 91%, 92%, 93%, 94%, 95%, 96%, 97%, 98%, 99% or more identical to a VH CDR3 sequence comprising the amino acid sequence GGRDRVPAVFDY (SEQ ID NO: 48); a VL CDR1 sequence that includes a sequence that is at least 90%, 91%, 92%, 93%, 94%, 95%, 96%, 97%, 98%, 99% or more identical to a VL CDR1 sequence comprising the amino acid sequence comprising the amino acid sequence RASQSIGTFLN (SEQ ID NO: 40); a VL CDR2 sequence that includes a sequence that is at least 90%, 91%, 92%, 93%, 94%, 95%, 96%, 97%, 98%, 99% or more identical to a VL CDR2 sequence comprising the amino acid sequence VASSLQS (SEQ ID NO: 41); and a VL CDR3 sequence that includes a sequence that is at least 90%, 91%, 92%, 93%, 94%, 95%, 96%, 97%, 98%, 99% or more identical to a VL CDR3 sequence comprising the amino acid sequence QQSYSVPIT (SEQ ID NO: 42).

In some embodiments, the antibody or antigen-binding fragment thereof comprises a combination of a variable heavy chain frame work region 1 (VH FR1) sequence, a variable heavy chain frame work region 2 (VH FR2) sequence, a variable heavy chain frame work region 3 (VH FR3) sequence, a variable heavy chain frame work region 4 (VH FR4) sequence, a variable light chain frame work region 1 (VL FR1) sequence, a variable light chain frame work region 2 (VL FR2) sequence, a variable light chain frame work region 3 (VL FR3) sequence, and a variable light chain frame work region 4 (VL FR4) sequence, wherein at least one frame work region (FR) sequence is selected from the group consisting of a VL FR1 sequence comprising the amino acid sequence DIQMTQSPSSLSASVGDRVTITC (SEQ ID NO: 49); a VL FR2 sequence comprising the amino acid sequence WYQQKPGKAPKVLIY (SEQ ID NO: 50); a VL FR3 sequence comprising the amino acid sequence GVPSRFSGSGSGTDFTLTISSLQPEDFATYYC (SEQ ID NO: 51); a VL FR4 sequence comprising the amino acid sequence FGQGTRLEIKR (SEQ ID NO: 52); a VH FR1 sequence comprising the amino acid sequence EVQLVESGGGLVQPGGSLRLSCAASGFTFS (SEQ ID NO: 57); a VH FR2 sequence comprising the amino acid sequence WVRQAPGKGLEYVS (SEQ ID NO: 58); a VH FR3 sequence comprising the amino acid sequence RFTISRDNSKNTLYLQMGSLRAEDMAVYYCAR (SEQ ID NO: 59); and a VH FR4 sequence comprising the amino acid sequence WGQGTMVTVSS (SEQ ID NO: 60).

In some embodiments, the antibody or antigen-binding fragment thereof comprises a combination of a variable heavy chain frame work region 1 (VH FR1) sequence, a variable heavy chain frame work region 2 (VH FR2) sequence, a variable heavy chain frame work region 3 (VH FR3) sequence, a variable heavy chain frame work region 4 (VH FR4) sequence, a variable light chain frame work region 1 (VL FR1) sequence, a variable light chain frame work region 2 (VL FR2) sequence, a variable light chain frame work region 3 (VL FR3) sequence, and a variable light chain frame work region 4 (VL FR4) sequence, wherein at least one frame work region (FR) sequence is selected from the group consisting of a VH FR1 sequence comprising the amino acid sequence EVQLVESGGGWVQPGGSLRLSCAASGFTFS (SEQ ID NO: 61); a VH FR2 sequence comprising the amino acid sequence WVRQAPGKGLEYVS (SEQ ID NO: 62); a VH FR3 sequence comprising the amino acid sequence RFTISRDNSKNLLYVHMGSLKPEDMAMYYCAR (SEQ ID NO: 63); a VH FR4 sequence comprising the amino acid sequence WGQGTLVTVSS (SEQ ID NO: 64); a VL FR1 sequence comprising the amino acid sequence DIQMTQSPSSLSASIGDRVTITC (SEQ ID NO: 53); a VL FR2 sequence comprising the amino acid sequence WYQQKPGKAPKVLIY (SEQ ID NO: 54); a VL FR3 sequence comprising the amino acid sequence GVPSRFIGSGSGTDFTLTISSLQPEDFATYYC (SEQ ID NO: 55); and a VL FR4 sequence comprising the amino acid sequence FGQGTRLEIKR (SEQ ID NO: 56).

In some embodiments, the antibody or antigen-binding fragment thereof is encoded by a nucleic acid sequence that comprises a nucleic acid sequence encoding a heavy chain amino acid sequence comprising SEQ ID NO: 67 or SEQ ID NO: 68. In some embodiments, the antibody or antigen-binding fragment thereof is encoded by a nucleic acid sequence that comprises a nucleic acid sequence encoding a heavy chain amino acid sequence comprising the amino acid sequence selected SEQ ID NO: 67. In some embodiments, the antibody or antigen-binding fragment thereof is encoded by a nucleic acid sequence that comprises a nucleic acid sequence encoding a heavy chain amino acid sequence comprising the amino acid sequence selected SEQ ID NO: 68.

In some embodiments, the antibody or antigen-binding fragment thereof is encoded by a nucleic acid sequence that comprises a nucleic acid sequence encoding a light chain amino acid sequence comprising SEQ ID NO: 67 or SEQ ID NO: 68. In some embodiments, the antibody or antigen-binding fragment thereof is encoded by a nucleic acid sequence that comprises a nucleic acid sequence encoding a light chain amino acid sequence comprising the amino acid sequence selected SEQ ID NO: 67. In some embodiments, the antibody or antigen-binding fragment thereof is encoded by a nucleic acid sequence that comprises a nucleic acid sequence encoding a light chain amino acid sequence comprising the amino acid sequence selected SEQ ID NO: 68.

In some embodiments, the antibody or antigen-binding fragment thereof is encoded by a nucleic acid sequence that comprises a nucleic acid sequence encoding a heavy chain amino acid sequence comprising the amino acid sequence of SEQ ID NO: 67, and a nucleic acid sequence encoding a light chain amino acid sequence comprising the amino acid sequence SEQ ID NO: 67. In some embodiments, the antibody or antigen-binding fragment thereof is encoded by a nucleic acid sequence that comprises a nucleic acid sequence encoding a heavy chain amino acid sequence comprising the amino acid sequence of SEQ ID NO. 68, and a nucleic acid sequence encoding a light chain amino acid sequence comprising the amino acid sequence SEQ ID NO: 68.

In some embodiments, the antibody or antigen-binding fragment thereof is encoded by a nucleic acid sequence that comprises a nucleic acid sequence that is at least 90%, 91%, 92%, 93%, 94%, 95%, 96%, 97%, 98% or 99% identical to a nucleic acid sequence encoding a heavy chain amino acid sequence comprising an amino acid sequence selected from the group consisting of SEQ ID NO: 67 or SEQ ID NO: 68. In some embodiments, the antibody or antigen-binding fragment thereof is encoded by a nucleic acid sequence that comprises a nucleic acid sequence that is at least 90%, 91%, 92%, 93%, 94%, 95%, 96%, 97%, 98% or 99% identical to a nucleic acid sequence encoding a heavy chain amino acid sequence comprising the amino acid sequence of SEQ ID NO: 67. In some embodiments, the antibody or antigen-binding fragment thereof is encoded by a nucleic acid sequence that comprises a nucleic acid sequence that is at least 90%, 91%, 92%, 93%, 94%, 95%, 96%, 97%, 98% or 99% identical to a nucleic acid sequence encoding a heavy chain amino acid sequence comprising the amino acid sequence of SEQ ID NO: 68.

In some embodiments, the antibody or antigen-binding fragment thereof is encoded by a nucleic acid sequence that comprises a nucleic acid sequence that is at least 90%, 91%, 92%, 93%, 94%, 95%, 96%, 97%, 98% or 99% identical to a nucleic acid sequence encoding a light chain amino acid sequence comprising an amino acid sequence selected from the group consisting of SEQ ID NO: 67 or SEQ ID NO: 68. In some embodiments, the antibody or antigen-binding fragment thereof is encoded by a nucleic acid sequence that comprises a nucleic acid sequence that is at least 90%, 91%, 92%, 93%, 94%, 95%, 96%, 97%, 98% or 99% identical to a nucleic acid sequence encoding a light chain amino acid sequence comprising the amino acid sequence of SEQ ID NO: 67. In some embodiments, the antibody or antigen-binding fragment thereof is encoded by a nucleic acid sequence that comprises a nucleic acid sequence that is at least 90%, 91%, 92%, 93%, 94%, 95%, 96%, 97%, 98% or 99% identical to a nucleic acid sequence encoding a light chain amino acid sequence comprising the amino acid sequence of SEQ ID NO: 68.

In some embodiments, the antibody or antigen-binding fragment thereof is encoded by a nucleic acid sequence that comprises a nucleic acid sequence that is at least 90%, 91%, 92%, 93%, 94%, 95%, 96%, 97%, 98% or 99% identical to a nucleic acid sequence encoding a heavy chain amino acid sequence comprising the amino acid sequence of SEQ ID NO: 67, and a nucleic acid sequence that is at least 90%, 91%, 92%, 93%, 94%, 95%, 96%, 97%, 98% or 99% identical to a nucleic acid sequence encoding a light chain amino acid sequence comprising the amino acid sequence of SEQ ID NO: 67. In some embodiments, the antibody or antigen-binding fragment thereof is encoded by a nucleic acid sequence that comprises a nucleic acid sequence that is at least 90%, 91%, 92%, 93%, 94%, 95%, 96%, 97%, 98% or 99% identical to a nucleic acid sequence encoding a heavy chain amino acid sequence comprising the amino acid sequence of SEQ ID NO: 68, and a nucleic acid sequence that is at least 90%, 91%, 92%, 93%, 94%, 95%, 96%, 97%, 98% or 99% identical to a nucleic acid sequence encoding a light chain amino acid sequence comprising the amino acid sequence of SEQ ID NO: 68.

In some embodiments, the antibody or antigen-binding fragment thereof is encoded by a nucleic acid sequence encoding a heavy chain amino acid sequence comprising the nucleic acid sequence SEQ ID NO: 71 or SEQ ID NO: 72. In some embodiments, the antibody or antigen-binding fragment thereof is encoded by a nucleic acid sequence encoding a heavy chain amino acid sequence comprising the nucleic acid sequence SEQ ID NO: 71. In some embodiments, the antibody or antigen-binding fragment thereof is encoded by a nucleic acid sequence encoding a heavy chain amino acid sequence comprising the nucleic acid sequence selected SEQ ID NO: 72.

In some embodiments, the antibody or antigen-binding fragment thereof is encoded by a nucleic acid sequence encoding a light chain amino acid sequence comprising the nucleic acid sequence SEQ ID NO: 69 or SEQ ID NO: 70. In some embodiments, the antibody or antigen-binding fragment thereof is encoded by a nucleic acid sequence encoding a light chain amino acid sequence comprising the nucleic acid sequence SEQ ID NO: 69. In some embodiments, the antibody or antigen-binding fragment thereof is encoded by a nucleic acid sequence encoding a light chain amino acid sequence comprising the nucleic acid sequence selected SEQ ID NO: 70.

In some embodiments, the antibody or antigen-binding fragment thereof is encoded by a nucleic acid sequence encoding a heavy chain amino acid sequence comprising the nucleic acid sequence of SEQ ID NO: 71, and a nucleic acid sequence encoding a light chain amino acid sequence comprising the nucleic acid sequence SEQ ID NO: 69. In some embodiments, the antibody or antigen-binding fragment thereof is encoded by a nucleic acid sequence encoding a heavy chain amino acid sequence comprising the nucleic acid sequence of SEQ ID NO: 72, and a nucleic acid sequence encoding a light chain amino acid sequence comprising the nucleic acid sequence SEQ ID NO: 70.

In some embodiments, the antibody or antigen-binding fragment thereof is incorporated in a multispecific antibody or antigen-binding fragment thereof, where at least one arm of the multispecific antibody or antigen-binding fragment thereof specifically binds SLC34A2. In some embodiments, the antibody or antigen-binding fragment thereof is incorporated in a bispecific antibody or antigen-binding fragment thereof, where at least one arm of the bispecific antibody or antigen-binding fragment thereof specifically binds SLC34A2.

In some embodiments, at least one arm of the multispecific antibody or antigen-binding fragment thereof, e.g., a bispecific antibody or antigen-binding fragment thereof, comprises a heavy chain variable region amino acid sequence comprising an amino acid sequence selected from the group consisting of SEQ ID NO: 67 or SEQ ID NO: 68. In some embodiments, at least one arm of the multispecific antibody or antigen-binding fragment thereof, e.g., a bispecific antibody or antigen-binding fragment thereof, comprises a heavy chain variable region amino acid sequence comprising the amino acid sequence of SEQ ID NO: 67. In some embodiments, at least one arm of the multispecific antibody or antigen-binding fragment thereof, e.g., a bispecific antibody or antigen-binding fragment thereof, comprises a heavy chain variable region amino acid sequence comprising the amino acid sequence of SEQ ID NO: 68.

In some embodiments, at least one arm of the multispecific antibody or antigen-binding fragment thereof, e.g., a bispecific antibody or antigen-binding fragment thereof, comprises a light chain variable region amino acid sequence comprising an amino acid sequence selected from the group consisting of SEQ ID NO: 67 or SEQ ID NO: 68. In some embodiments, at least one arm of the multispecific antibody or antigen-binding fragment thereof, e.g., a bispecific antibody or antigen-binding fragment thereof, comprises a light chain variable region amino acid sequence comprising an amino acid sequence of SEQ ID NO: 67. In some embodiments, at least one arm of the multispecific antibody or antigen-binding fragment thereof, e.g., a bispecific antibody or antigen-binding fragment thereof, comprises a light chain variable region amino acid sequence comprising the amino acid sequence of SEQ ID NO: 68.

In some embodiments, at least one arm of the multispecific antibody or antigen-binding fragment thereof, e.g., a bispecific antibody or antigen-binding fragment thereof, comprises a heavy chain variable region amino acid sequence comprising an amino acid sequence selected from the group consisting of SEQ ID NO: 67 and SEQ ID NO: 68, and a light chain variable region amino acid sequence comprising an amino acid sequence selected from the group consisting of SEQ ID NO: 67 and SEQ ID NO: 68.

In some embodiments, at least one arm of the multispecific antibody or antigen-binding fragment thereof, e.g., a bispecific antibody or antigen-binding fragment thereof, comprises a heavy chain variable region amino acid sequence comprising an amino acid sequence selected from the group consisting of SEQ ID NO: 67 and SEQ ID NO: 68, and a light chain variable region amino acid sequence comprising an amino acid sequence selected from the group consisting of SEQ ID NO: 67 and SEQ ID NO: 68. In some embodiments, at least one arm of the multispecific antibody or antigen-binding fragment thereof, e.g., a bispecific antibody or antigen-binding fragment thereof, comprises a heavy chain variable region amino acid sequence comprising the amino acid sequence of SEQ ID NO: SEQ ID NO: 67, and a light chain variable region amino acid sequence comprising the amino acid sequence of SEQ ID NO: 67, a bispecific antibody or antigen-binding fragment thereof, comprises a heavy chain variable region amino acid sequence comprising the amino acid sequence of SEQ ID NO: SEQ ID NO: 68, and a light chain variable region amino acid sequence comprising the amino acid sequence of SEQ ID NO: 68.

In some embodiments, at least one arm of the multispecific antibody or antigen-binding fragment thereof, e.g., a bispecific antibody or antigen-binding fragment thereof, comprises a heavy chain variable region amino acid sequence that is at least 90%, 91%, 92%, 93%, 94%, 95%, 96%, 97%, 98% or 99% identical to an amino acid sequence comprising an amino acid sequence selected from the group consisting of SEQ ID NO: 67 and SEQ ID NO: 68. In some embodiments, at least one arm of the multispecific antibody or antigen-binding fragment thereof, e.g., a bispecific antibody or antigen-binding fragment thereof, comprises a heavy chain variable region amino acid sequence that is at least 90%, 91%, 92%, 93%, 94%, 95%, 96%, 97%, 98% or 99% identical to an amino acid sequence comprising the amino acid sequence of SEQ ID NO: 67. In some embodiments, at least one arm of the multispecific antibody or antigen-binding fragment thereof, e.g., a bispecific antibody or antigen-binding fragment thereof, comprises a heavy chain variable region amino acid sequence that is at least 90%, 91%, 92%, 93%, 94%, 95%, 96%, 97%, 98% or 99% identical to an amino acid sequence comprising the amino acid sequence of SEQ ID NO: 68.

In some embodiments, at least one arm of the multispecific antibody or antigen-binding fragment thereof, e.g., a bispecific antibody or antigen-binding fragment thereof, comprises a light chain variable region amino acid sequence that is at least 90%, 91%, 92%, 93%, 94%, 95%, 96%, 97%, 98% or 99% identical to an amino acid sequence comprising an amino acid sequence selected from the group consisting of SEQ ID NO: 67 and SEQ ID NO: 68. In some embodiments, at least one arm of the multispecific antibody or antigen-binding fragment thereof, e.g., a bispecific antibody or antigen-binding fragment thereof, comprises a light chain variable region amino acid sequence that is at least 90%, 91%, 92%, 93%, 94%, 95%, 96%, 97%, 98% or 99% identical to an amino acid sequence comprising an amino acid sequence of SEQ ID NO. 67. In some embodiments, at least one arm of the multispecific antibody or antigen-binding fragment thereof, e.g., a bispecific antibody or antigen-binding fragment thereof, comprises a light chain variable region amino acid sequence that is at least 90%, 91%, 92%, 93%, 94%, 95%, 96%, 97%, 98% or 99% identical to an amino acid sequence comprising the amino acid sequence of SEQ ID NO: 68.

In some embodiments, at least one arm of the multispecific antibody or antigen-binding fragment thereof, e.g., a bispecific antibody or antigen-binding fragment thereof, comprises a heavy chain variable region amino acid sequence that is at least 90%, 91%, 92%, 93%, 94%, 95%, 96%, 97%, 98% or 99% identical to an amino acid sequence of SEQ ID NO: 67, and a light chain variable region amino acid sequence that is at least 90%, 91%, 92%, 93%, 94%, 95%, 96%, 97%, 98% or 99% identical to an amino acid sequence comprising of SEQ ID NO: 67.

In some embodiments, at least one arm of the multispecific antibody or antigen-binding fragment thereof, e.g., a bispecific antibody or antigen-binding fragment thereof, comprises a heavy chain variable region amino acid sequence that is at least 90%, 91%, 92%, 93%, 94%, 95%, 96%, 97%, 98% or 99% identical to an amino acid sequence of SEQ ID NO: 68, and a light chain variable region amino acid sequence that is at least 90%, 91%, 92%, 93%, 94%, 95%, 96%, 97%, 98% or 99% identical to an amino acid sequence comprising of SEQ ID NO: 68.

In some embodiments, at least one arm of the multispecific antibody or antigen-binding fragment thereof, e.g., a bispecific antibody or antigen-binding fragment thereof, comprises a combination of a variable heavy chain complementarity determining region 1 (VH CDR1, also referred to herein as CDRH1) sequence, a variable heavy chain complementarity determining region 2 (VH CDR2, also referred to herein as CDRH2) sequence, a variable heavy chain complementarity determining region 3 (VH CDR3, also referred to herein as CDRH3) sequence, a variable light chain complementarity determining region 1 (VL CDR1, also referred to herein as CDRL1) sequence, a variable light chain complementarity determining region 2 (VL CDR2, also referred to herein as CDRL2) sequence, and a variable light chain complementarity determining region 3 (VL CDR3, also referred to herein as CDRL3) sequence, wherein at least one complementarity determining region (CDR) sequence is selected from the group consisting of a VH CDR1 sequence comprising the amino acid sequence SYVMH (SEQ ID NO: 43) or SHIMY (SEQ ID NO: 46); a VH CDR2 sequence comprising the amino acid sequence GVSSSGDSTFYVDSVKG (SEQ ID NO: 44) or GISSNGLSSYYVDSVKG (SEQ ID NO: 47); a VH CDR3 sequence comprising the amino acid sequence GGITGAPLVFDI (SEQ ID NO: 45) or GGRDRVPAVFDY (SEQ ID NO: 48); a VL CDR1 sequence comprising the amino acid sequence RASQSISRFLN (SEQ ID NO: 37) or RASQSIGTFLN (SEQ ID NO: 40); a VL CDR2 sequence comprising the amino acid sequence VTSSLQS (SEQ ID NO: 38) or VASSLQS (SEQ ID NO: 41); and a VL CDR3 sequence comprising the amino acid sequence QQSYNTPIT (SEQ ID NO: 39) or QQSYSVPIT (SEQ ID NO: 42).

In some embodiments, at least one arm of the multispecific antibody or antigen-binding fragment thereof, e.g., a bispecific antibody or antigen-binding fragment thereof, comprises a combination of a VH CDR1 sequence, a VH CDR2 sequence, a VH CDR3 sequence, a VL CDR1 sequence, a VL CDR2 sequence, and a VL CDR3 sequence, wherein at one complementarity determining region (CDR) sequence is selected from the group consisting of a VH CDR1 sequence comprising the amino acid sequence SYVMH (SEQ ID NO: 43); a VH CDR2 sequence comprising the amino acid sequence GVSSSGDSTFYVDSVKG (SEQ ID NO: 44); a VH CDR3 sequence comprising the amino acid sequence GGITGAPLVFDI (SEQ ID NO: 45); a VL CDR1 sequence comprising the amino acid sequence RASQSISRFLN (SEQ ID NO: 37): a VL CDR2 sequence comprising the amino acid sequence VTSSLQS (SEQ ID NO: 38); and a VL CDR3 sequence comprising the amino acid sequence QQSYNTPIT (SEQ ID NO: 39).

In some embodiments, at least one arm of the multispecific antibody or antigen-binding fragment thereof, e.g., a bispecific antibody or antigen-binding fragment thereof, comprises a combination of a VH CDR1 sequence, a VH CDR2 sequence, a VH CDR3 sequence, a VL CDR1 sequence, a VL CDR2 sequence, and a VL CDR3 sequence, wherein at least one CDR sequence is selected from the group consisting of a VH CDR1 sequence that includes a sequence that is at least 90%, 91%, 92%, 93%, 94%, 95%, 96%, 97%, 98%, 99% or more identical to a VH CDR1 sequence comprising the amino acid sequence SYVMH (SEQ ID NO: 43); a VH CDR2 sequence that includes a sequence that is at least 90%, 91%, 92%, 93%, 94%, 95%, 96%, 97%, 98%, 99% or more identical to a VH CDR2 sequence comprising the amino acid sequence GVSSSGDSTFYVDSVKG (SEQ ID NO: 44); a VH CDR3 sequence that includes a sequence that is at least 90%, 91%, 92%, 93%, 94%, 95%, 96%, 97%, 98%, 99% or more identical to a VH CDR3 sequence comprising the amino acid sequence GGITGAPLVFDI (SEQ ID NO: 45); a VL CDR1 sequence that includes a sequence that is at least 90%, 91%, 92%, 93%, 94%, 95%, 96%, 97%, 98%, 99% or more identical to a VL CDR1 sequence comprising the amino acid sequence comprising the amino acid sequence RASQSISRFLN (SEQ ID NO: 37); a VL CDR2 sequence that includes a sequence that is at least 90%, 91%, 92%, 93%, 94%, 95%, 96%, 97%, 98%, 99% or more identical to a VL CDR2 sequence comprising the amino acid sequence VTSSLQS (SEQ ID NO: 38); and a VL CDR3 sequence that includes a sequence that is at least 90%, 91%, 92%, 93%, 94%, 95%, 96%, 97%, 98%, 99% or more identical to a VL CDR3 sequence comprising the amino acid sequence QQSYNTPIT (SEQ ID NO: 39).

In some embodiments, at least one arm of the multispecific antibody or antigen-binding fragment thereof, e.g., a bispecific antibody or antigen-binding fragment thereof, comprises a combination of a VH CDR1 sequence, a VH CDR2 sequence, a VH CDR3 sequence, a VL CDR1 sequence, a VL CDR2 sequence, and a VL CDR3 sequence, wherein at least one complementarity determining region (CDR) sequence is selected from the group consisting of a VH CDR1 sequence comprising the amino acid sequence SHIMY (SEQ ID NO: 46); a VH CDR2 sequence comprising the amino acid sequence GISSNGLSSYYVDSVKG (SEQ ID NO: 47); a VH CDR3 sequence comprising the amino acid sequence GGRDRVPAVFDY (SEQ ID NO: 48); a VL CDR1 sequence comprising the amino acid sequence RASQSIGTFLN (SEQ ID NO: 40); a VL CDR2 sequence comprising the amino acid sequence VASSLQS (SEQ ID NO: 41); and a VL CDR3 sequence comprising the amino acid sequence QQSYSVPIT (SEQ ID NO: 42).

In some embodiments, at least one arm of the multispecific antibody or antigen-binding fragment thereof, e.g., a bispecific antibody or antigen-binding fragment thereof, comprises a combination of a VH CDR1 sequence, a VH CDR2 sequence, a VH CDR3 sequence, a VL CDR1 sequence, a VL CDR2 sequence, and a VL CDR3 sequence, wherein at least one CDR sequence is selected from the group consisting of a VH CDR1 sequence that includes a sequence that is at least 90%, 91%, 92%, 93%, 94%, 95%, 96%, 97%, 98%, 99% or more identical to a VH CDR1 sequence comprising the amino acid sequence SHIMY (SEQ ID NO: 46); a VH CDR2 sequence that includes a sequence that is at least 90%, 91%, 92%, 93%, 94%, 95%, 96%, 97%, 98%, 99% or more identical to a VH CDR2 sequence comprising the amino acid sequence GISSNGLSSYYVDSVKG (SEQ ID NO: 47); a VH CDR3 sequence that includes a sequence that is at least 90%, 91%, 92%, 93%, 94%, 95%, 96%, 97%, 98%, 99% or more identical to a VH CDR3 sequence comprising the amino acid sequence GGRDRVPAVFDY (SEQ ID NO: 48); a VL CDR1 sequence that includes a sequence that is at least 90%, 91%, 92%, 93%, 94%, 95%, 96%, 97%, 98%, 99% or more identical to a VL CDR1 sequence comprising the amino acid sequence comprising the amino acid sequence RASQSIGTFLN (SEQ ID NO: 40); a VL CDR2 sequence that includes a sequence that is at least 90%, 91%, 92%, 93%, 94%, 95%, 96%, 97%, 98%, 99% or more identical to a VL CDR2 sequence comprising the amino acid sequence VASSLQS (SEQ ID NO: 41); and a VL CDR3 sequence that includes a sequence that is at least 90%, 91%, 92%, 93%, 94%, 95%, 96%, 97%, 98%, 99% or more identical to a VL CDR3 sequence comprising the amino acid sequence QQSYSVPIT (SEQ ID NO: 42).

Suitable anti-SLC34A2 antibodies of the disclosure also include an antibody or antigen binding fragment thereof that binds to the same epitope on human SLC34A2 and/or cynomolgus monkey SLC34A2 as an anti-SLC34A2 antibody comprising a heavy chain variable region amino acid sequence selected from the group consisting of SEQ ID NO: 67 and SEQ ID NO: 68, and a light chain variable region amino acid sequence selected from the group consisting of SEQ ID NO: 67 and SEQ ID NO: 68.

Suitable anti-SLC34A2 antibodies of the disclosure also include an antibody or antigen binding fragment thereof that binds to the same epitope on human SLC34A2 and/or cynomolgus monkey SLC34A2 as an anti-SLC34A2 antibody comprises the VH CDR1 sequence comprising the amino acid sequence SYVMH (SEQ ID NO: 43); the VH CDR2 sequence comprising the amino acid sequence GVSSSGDSTFYVDSVKG (SEQ ID NO: 44); the VH CDR3 sequence comprising the amino acid sequence GGITGAPLVFDI (SEQ ID NO: 45); the VL CDR1 sequence comprising the amino acid sequence RASQSISRFLN (SEQ ID NO: 37); the VL CDR2 sequence comprising the amino acid sequence VTSSLQS (SEQ ID NO: 38); and the VL CDR3 sequence comprising the amino acid sequence QQSYNTPIT (SEQ ID NO: 39).

Suitable anti-SLC34A2 antibodies of the disclosure also include an antibody or antigen binding fragment thereof that binds to the same epitope on human SLC34A2 and/or cynomolgus monkey SLC34A2 as an anti-SLC34A2 antibody comprises the VH CDR1 sequence comprising the amino acid sequence SHIMY (SEQ ID NO: 46); the VH CDR2 sequence comprising the amino acid sequence GISSNGLSSYYVDSVKG (SEQ ID NO: 47); the VH CDR3 sequence comprising the amino acid sequence GGRDRVPAVFDY (SEQ ID NO: 48); the VL CDR1 sequence comprising the amino acid sequence RASQSIGTFLN (SEQ ID NO: 40); the VL CDR2 sequence comprising the amino acid sequence VASSLQS (SEQ ID NO: 41); and the VL CDR3 sequence comprising the amino acid sequence QQSYSVPIT (SEQ ID NO: 42).

Suitable anti-SLC34A2 antibodies of the disclosure also include an antibody or antigen binding fragment thereof that cross-competes for binding to human SLC34A2 and/or cynomolgus monkey SLC34A2 as an anti-SLC34A2 antibody comprising a heavy chain variable region amino acid sequence selected from the group consisting of SEQ ID NO: 67 and SEQ ID NO: 68, and a light chain variable region amino acid sequence selected from the group consisting of SEQ ID NO: 67 and SEQ ID NO: 68.

Suitable anti-SLC34A2 antibodies of the disclosure also include an antibody or antigen binding fragment thereof that cross-competes for binding to human SLC34A2 and/or cynomolgus monkey SLC34A2 as an anti-SLC34A2 antibody comprises the VH CDR1 sequence comprising the amino acid sequence SYVMH (SEQ ID NO: 43); the VH CDR2 sequence comprising the amino acid sequence GVSSSGDSTFYVDSVKG (SEQ ID NO: 44); the VH CDR3 sequence comprising the amino acid sequence GGITGAPLVFDI (SEQ ID NO: 45), the VL CDR1 sequence comprising the amino acid sequence RASQSISRFLN (SEQ ID NO: 37); the VL CDR2 sequence comprising the amino acid sequence VTSSLQS (SEQ ID NO: 38); and the VL CDR3 sequence comprising the amino acid sequence QQSYNTPIT (SEQ ID NO: 39).

Suitable anti-SLC34A2 antibodies of the disclosure also include an antibody or antigen binding fragment thereof that cross-competes for binding to human SLC34A2 and/or cynomolgus monkey SLC34A2 as an anti-SLC34A2 antibody comprises the VH CDR1 sequence comprising the amino acid sequence SHIMY (SEQ ID NO: 46); the VH CDR2 sequence comprising the amino acid sequence GISSNGLSSYYVDSVKG (SEQ ID NO: 47); the VH CDR3 sequence comprising the amino acid sequence GGRDRVPAVFDY (SEQ ID NO: 48); the VL CDR1 sequence comprising the amino acid sequence RASQSIGTFLN (SEQ ID NO: 40); the VL CDR2 sequence comprising the amino acid sequence VASSLQS (SEQ ID NO: 41); and the VL CDR3 sequence comprising the amino acid sequence QQSYSVPIT (SEQ ID NO: 42).

The invention also provides methods of treating, preventing and/or delaying the onset or progression of, or alleviating a symptom associated with aberrant expression and/or activity of SLC34A2 in a subject using activatable antibodies that bind SLC34A2, particularly activatable antibodies that bind and neutralize or otherwise inhibit at least one biological activity of SLC34A2 and/or SLC34A2-mediated signaling.

The invention also provides methods of treating, preventing and/or delaying the onset or progression of, or alleviating a symptom associated with the presence, growth, proliferation, metastasis, and/or activity of cells which are expressing SLC34A2 or aberrantly expressing SLC34A2 in a subject that bind, target, neutralize, kill, or otherwise inhibit at least one biological activity of cells which are expressing or aberrantly expressing SLC34A2.

The invention also provides methods of treating, preventing and/or delaying the onset or progression of, or alleviating a symptom associated with the presence, growth, proliferation, metastasis, and/or activity of cells which are expressing SLC34A2 in a subject that bind, target, neutralize, kill, or otherwise inhibit at least one biological activity of cells which are expressing SLC34A2.

The invention also provides methods of treating, preventing and/or delaying the onset or progression of, or alleviating a symptom associated with the presence, growth, proliferation, metastasis, and/or activity of cells which are aberrantly expressing SLC34A2 in a subject that bind, target, neutralize, kill, or otherwise inhibit at least one biological activity of cells which are aberrantly expressing SLC34A2.

In some embodiments, the mammalian SLC34A2 is selected from the group consisting of a human SLC34A2 and a cynomolgus monkey SLC34A2. In some embodiments, the AB specifically binds to human SLC34A2 or cynomolgus monkey SLC34A2 with a dissociation constant of less than 1 nM. In some embodiments, the mammalian SLC34A2 is a human SLC34A2. In some embodiments, the mammalian SLC34A2 is a cynomolgus SLC34A2.

In some embodiments, the AB has one or more of the following characteristics: (a) the AB specifically binds to human SLC34A2; and (b) the AB specifically binds to human SLC34A2 and cynomolgus monkey SLC34A2.

In some embodiments, the AB blocks the ability of a natural ligand or receptor to bind to the mammalian SLC34A2 with an EC50 less than or equal to 5 nM, less than or equal to 10 nM, less than or equal to 50 nM, less than or equal to 100 nM, less than or equal to 500 nM, and/or less than or equal to 1000 nM.

In some embodiments, the AB blocks the ability of a natural ligand to bind to the mammalian SLC34A2 with an EC50 of 5 nM to 1000 nM, 5 nM to 500 nM, 5 nM to 100 nM 5 nM to 50 nM, 5 nM to 10 nM, 10 nM to 1000 nM, 10 nM to 500 nM, 10 nM to 100 nM 10 nM to 50 nM, 50 nM to 1000 nM, 50 nM to 500 nM, 50 nM to 100 nM, 100 nM to 1000 nM, 100 nM to 500 nM, 500 nM to 1000 nM.

In some embodiments, the AB of the present disclosure inhibits or reduces the growth, proliferation, and/or metastasis of cells expressing mammalian SLC34A2. Without intending to be bound by any theory, the AB of the present disclosure may inhibit or reduce the growth, proliferation, and/or metastasis of cells expressing mammalian SLC34A2 by specifically binding to SLC34A2 and inhibiting, blocking, and/or preventing the binding of a natural ligand or receptor to mammalian SLC34A2.

In some embodiments, the antibody includes an agent conjugated to the AB. In some embodiments, the agent conjugated to the AB or the AB of an antibody is a therapeutic agent. In some embodiments, the agent is an antineoplastic agent. In some embodiments, the agent is a toxin or fragment thereof. As used herein, a fragment of a toxin is a fragment that retains toxic activity. In some embodiments, the agent is conjugated to the AB via a cleavable linker. In some embodiments, the agent is conjugated to the AB via a noncleavable linker. In some embodiments, the agent is conjugated to the AB via a linker that is cleavable in an intracellular or lysosomal environment. In some embodiments, the agent is a microtubule inhibitor. In some embodiments, the agent is a nucleic acid damaging agent, such as a DNA alkylator, a DNA cleaving agent, a DNA cross-linker, a DNA intercalator, or other DNA damaging agent. In some embodiments, the agent is an agent selected from the group listed in Table 5. In some embodiments, the agent is a dolastatin. In some embodiments, the agent is an auristatin or derivative thereof. In some embodiments, the agent is auristatin E or a derivative thereof. In some embodiments, the agent is monomethyl auristatin E (MMAE). In some embodiments, the agent is monomethyl auristatin D (MMAD). In some embodiments, the agent is a maytansinoid or maytansinoid derivative. In some embodiments, the agent is DM1 or DM4. In some embodiments, the agent is a duocarmycin or derivative thereof. In some embodiments, the agent is a calicheamicin or derivative thereof. In some embodiments, the agent is a pyrrolobenzodiazepine. In some embodiments, the agent is a pyrrolobenzodiazepine dimer.

In some embodiments, the agent comprises a molecule having a structure of formula (I):

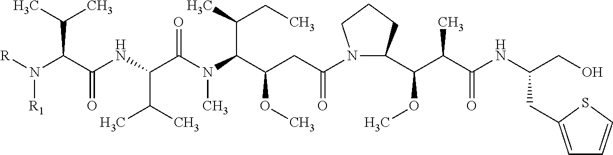

wherein R1 is a hydrogen or a C₁₋₆ alkyl group: wherein R is selected from the group consisting of: a hydrogen, a C₁₋₆ alkyl, a linker, or a group X1-Y1-* wherein * is the point of attachment to the nitrogen; and wherein Y1 is an oxycarbonyl group and X1 is a C₁₋₆ alkyl group, a 9-fluorenylmethyl group, a benzyl group, or a tert-butyl group In some embodiments, the antibody is conjugated to one or more equivalents of an agent.

In some embodiments, the agent is conjugated to the antibody via a linker having a structure of formula (II):

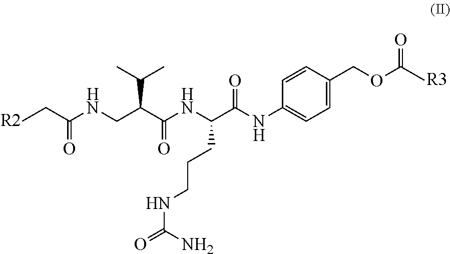

wherein R3 is an agent attached to formula (II) where the point of attachment is a nitrogen, sulfur, oxygen, or carbon atom; and wherein R2 is an moiety attached to formula (H) wherein the point of attachment is selected from the group consisting of: a chlorine group, a iodine group, a bromine group, and a thiol group.

In some embodiments, the agent is conjugated to the antibody via a linker, wherein the agent and linker has a structure of formula (III):

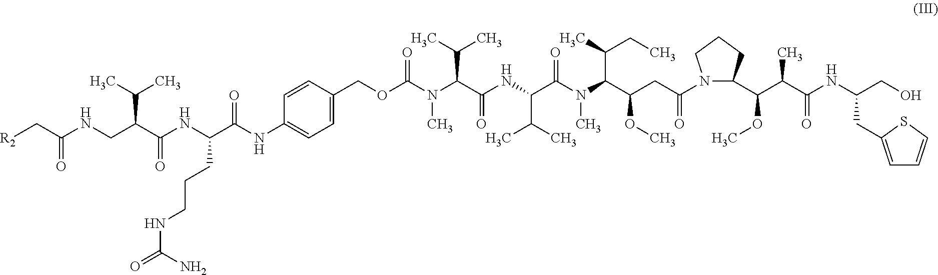

wherein R2 is a point of attachment to the AB.

In some embodiments, the antibody is conjugated to one equivalent of the agent. In some embodiments, the antibody is conjugated to two, three, four, five, six, seven, eight, nine, ten, or greater than ten equivalents of the agent. In some embodiments, the antibody is part of a mixture of antibodies having a homogeneous number of equivalents of conjugated agents. In some embodiments, the antibody is part of a mixture of antibodies having a heterogeneous number of equivalents of conjugated agents. In some embodiments, the mixture of antibodies is such that the average number of agents conjugated to each antibody is between zero to one, between one to two, between two and three, between three and four, between four and five, between five and six, between six and seven, between seven and eight, between eight and nine, between nine and ten, and ten and greater. In some embodiments, the mixture of antibodies is such that the average number of agents conjugated to each antibody is one, two, three, four, five, six, seven, eight, nine, ten, or greater. In some embodiments, the antibody comprises one or more site-specific amino acid sequence modifications such that the number of lysine and/or cysteine residues is increased or decreased with respect to the original amino acid sequence of the antibody, thus in some embodiments correspondingly increasing or decreasing the number of agents that can be conjugated to the antibody, or in some embodiments limiting the conjugation of the agents to the antibody in a site-specific manner. In some embodiments, the modified antibody is modified with one or more non-natural amino acids in a site-specific manner, thus in some embodiments limiting the conjugation of the agents to only the sites of the non-natural amino acids.

In some embodiments, the agent is an anti-inflammatory agent. In some embodiments, the antibody also includes a detectable moiety. In some embodiments, the detectable moiety is a diagnostic agent.

In some embodiments, the AB of the antibody naturally contains one or more disulfide bonds. In some embodiments, the AB can be engineered to include one or more disulfide bonds.

In some embodiments, the antibody drug conjugates (ADCs) can include one or more polypeptides that include the combination of a light chain sequence or a light chain variable domain sequence, and a heavy chain sequence or a heavy chain variable domain sequences, a linker, and a toxin.

In some embodiments, the anti-SLC34A2 antibody, or anti-SLC34A2 conjugated antibody is administered during and/or after treatment in combination with one or more additional agents such as, for example, a chemotherapeutic agent, an anti-inflammatory agent, and/or an immunosuppressive agent. In some embodiments, the anti-SLC34A2 antibody or conjugated anti-SLC34A2 antibody, and the additional agent are formulated into a single therapeutic composition, and the anti-SLC34A2 antibody or conjugated anti-SLC34A2 antibody, and additional agent are administered simultaneously. Alternatively, the anti-SLC34A2 antibody or conjugated anti-SLC34A2 antibody, and additional agent are separate from each other, e.g., each is formulated into a separate therapeutic composition, and the anti-SLC34A2 antibody or conjugated anti-SLC34A2 antibody, and the additional agent are administered simultaneously, or the anti-SLC34A2 antibody or conjugated anti-SLC34A2 antibody, and the additional agent are administered at different times during a treatment regimen. For example, the anti-SLC34A2 antibody or conjugated anti-SLC34A2 antibody, is administered prior to the administration of the additional agent, the anti-SLC34A2 antibody or conjugated anti-SLC34A2 antibody, is administered subsequent to the administration of the additional agent, or the anti-SLC34A2 antibody or conjugated anti-SLC34A2 antibody, and the additional agent are administered in an alternating fashion. As described herein, the anti-SLC34A2 antibody or conjugated anti-SLC34A2 antibody, and additional agent are administered in single doses or in multiple doses.

In some embodiments, the anti-SLC34A2 antibody or conjugated anti-SLC34A2 antibody, and the additional agent(s) are administered simultaneously. For example, the anti-SLC34A2 antibody or conjugated anti-SLC34A2 antibody, and the additional agent(s) can be formulated in a single composition or administered as two or more separate compositions. In some embodiments, the anti-SLC34A2 antibody or conjugated anti-SLC34A2 antibody, and the additional agent(s) are administered sequentially, or the anti-SLC34A2 antibody or conjugated anti-SLC34A2 antibody, and the additional agent are administered at different times during a treatment regimen.

In some embodiments, the anti-SLC34A2 antibody or conjugated anti-SLC34A2 antibody, is administered during and/or after treatment in combination with one or more additional agents such as, by way of non-limiting example, a chemotherapeutic agent, an anti-inflammatory agent, and/or an immunosuppressive agent, such as an alkylating agent, an anti-metabolite, an anti-microtubule agent, a topoisomerase inhibitor, a cytotoxic antibiotic, and/or any other nucleic acid damaging agent. In some embodiments, the additional agent is a taxane, such as paclitaxel (e.g., Abraxane®). In some embodiments, the additional agent is an anti-metabolite, such as gemcitabine. In some embodiments, the additional agent is an alkylating agent, such as platinum-based chemotherapy, such as carboplatin or cisplatin. In some embodiments, the additional agent is a targeted agent, such as a kinase inhibitor, e.g., sorafenib or erlotinib. In some embodiments, the additional agent is a targeted agent, such as another antibody, e.g., a monoclonal antibody (e.g., bevacizumab), a bispecific antibody, or a multispecific antibody. In some embodiments, the additional agent is a proteosome inhibitor, such as bortezomib or carfilzomib. In some embodiments, the additional agent is an immune modulating agent, such as lenolidominde or IL-2. In some embodiments, the additional agent is radiation. In some embodiments, the additional agent is an agent considered standard of care by those skilled in the art. In some embodiments, the additional agent is a chemotherapeutic agent well known to those skilled in the art.

In some embodiments, the additional agent is another antibody or antigen-binding fragment thereof, another conjugated antibody or antigen-binding fragment thereof, another activatable antibody or antigen-binding fragment thereof and/or another conjugated activatable antibody or antigen-binding fragment thereof. In some embodiments the additional agent is another antibody or antigen-binding fragment thereof, another conjugated antibody or antigen-binding fragment thereof, another activatable antibody or antigen-binding fragment thereof and/or another conjugated activatable antibody or antigen-binding fragment thereof against the same target as the first antibody or antigen-binding fragment thereof, the first conjugated antibody or antigen-binding fragment thereof, activatable antibody or antigen-binding fragment thereof and/or a conjugated activatable antibody or antigen-binding fragment thereof, e.g., against SLC34A2. In some embodiments the additional agent is another antibody or antigen-binding fragment thereof, another conjugated antibody or antigen-binding fragment thereof, another activatable antibody or antigen-binding fragment thereof and/or another conjugated activatable antibody or antigen-binding fragment thereof against a target different than the target of the first antibody or antigen-binding fragment thereof, the first conjugated antibody or antigen-binding fragment thereof, activatable antibody or antigen-binding fragment thereof and/or a conjugated activatable antibody or antigen-binding fragment thereof.

In some embodiments, the additional antibody or antigen binding fragment thereof, conjugated antibody or antigen binding fragment thereof, activatable antibody or antigen binding fragment thereof, and/or conjugated activatable antibody or antigen binding fragment thereof is a monoclonal antibody, domain antibody, single chain, Fab fragment, a F(ab′)₂ fragment, a scFv, a scAb, a dAb, a single domain heavy chain antibody, or a single domain light chain antibody. In some embodiments, the additional antibody or antigen binding fragment thereof, conjugated antibody or antigen binding fragment thereof, activatable antibody or antigen binding fragment thereof, and/or conjugated activatable antibody or antigen binding fragment thereof is a mouse, other rodent, chimeric, humanized or fully human monoclonal antibody.

The disclosure also provides methods of producing an anti-SLC34A2 antibody polypeptide by culturing a cell under conditions that lead to expression of the polypeptide, wherein the cell comprises an isolated nucleic acid molecule encoding an antibody described herein, and/or vectors that include these isolated nucleic acid sequences. The disclosure provides methods of producing an antibody by culturing a cell under conditions that lead to expression of the antibody, wherein the cell comprises an isolated nucleic acid molecule encoding an antibody described herein, and/or vectors that include these isolated nucleic acid sequences.

The invention also provides a method of manufacturing antibodies that binds SLC34A2 by (a) culturing a cell comprising a nucleic acid construct that encodes the antibody under conditions that lead to expression of the antibody, wherein the antibody or the antigen binding fragment thereof (AB) specifically binds SLC34A2; and (b) recovering the antibody.

The invention provides methods of preventing, delaying the progression of, treating, alleviating a symptom of, or otherwise ameliorating an SLC34A2 mediated disease in a subject by administering a therapeutically effective amount of an anti-SLC34A2 antibody, and/or conjugated anti-SLC34A2 antibody described herein to a subject in need thereof.

The invention also provides methods of preventing, delaying the progression of, treating, alleviating a symptom of, or otherwise ameliorating cancer in a subject by administering a therapeutically effective amount of an anti-SLC34A2 antibody, and/or conjugated anti-SLC34A2 antibody described herein to a subject in need thereof. Prostate-specific membrane antigen (SLC34A2) is a type 2 transmembrane glycoprotein with high and restricted expression in all forms of prostate tissue, including carcinoma. Studies have consistently demonstrated SLC34A2 expression in all types of prostate tissue and increased SLC34A2 expression in cancer tissue. SLC34A2 is also expressed in other cancers, more specifically in the neovasculature associated with these cancers.

### BRIEF DESCRIPTION OF THE DRAWINGS

FIG. 1 presents the Tms of the two anti-SLC34A2 antibodies.

FIG. 2 presents the two anti-SLC34A2 antibodies and ADCs do not bind to other SLC34 Family Members.

FIGS. 3a and 3b presents PK studies indicating the stability of the two conjugated anti-SLC34A2 antibodies, OT1 is more stable than OT2.

FIG. 4 presents in vitro cytotoxicity of the two conjugated anti-SLC34A2 antibodies in target-positive OVCAR3 cells. OT #1 and OT #2 kill target-positive OVCAR3 cells with high potency, with more than 10× better EC50 compared to the benchmark ADC. OT #1 has less off-target activity than OT #2.

FIGS. 5a and 5b presents in vivo toxicity of the two conjugated anti-SLC34A2 antibodies in an Ovarian OVCAR3 mouse model.

FIG. 6 presents in vivo toxicity of the two conjugated anti-SLC34A2 antibodies in an Ovarian PDX Model AG-OV37 mouse model

FIG. 7 presents OT #1 Inhibits Growth Of Human Lung Cancer Model NCI-H441.

FIG. 8 presents OT #1 Inhibits Growth Of Human Lung Cancer Model RERF-LC-Ad1.

FIG. 9 presents OT #1 Exhibits Favorable PK Properties In Rats.

### DETAILED DESCRIPTION OF THE INVENTION

The present disclosure provides monoclonal antibodies (mAbs) and anti-SLC34A2 drug conjugates that specifically bind SLC34A2.

In some embodiments, a target-binding moiety to which compounds of the present disclosure can be conjugated include anti-SLC34A2 antibodies, examples of which are described in the sequences below:

TABLE 9
VL CDR Amino Acid Sequences

| | VL CDR1 | VL CDR2 | VL CDR3 |
|---|---|---|---|
| Antibody | (SEQ ID NO:) | (SEQ ID NO:) | (SEQ ID NO:) |
| [Ag]AB-2 | SEQUENCE | SEQUENCE | SEQUENCE |
| cHv83-3a23.G1 | (SEQ ID NO: 37) | (SEQ ID NO: 38) | (SEQ ID NO: 39) |
| (L328C)k | RASQSISRFLN | VTSSLQS | QQSYNTPIT |
| [Ag]AB-3 | SEQUENCE | SEQUENCE | SEQUENCE |
| cHv83-1b15.G1 | (SEQ ID NO: 40) | (SEQ ID NO: 41) | (SEQ ID NO: 42) |
| (L328C)k | RASQSIGTFLN | VASSLQS | QQSYSVPIT |

TABLE 10
VH CDR Amino Acid Sequences

| | VH CDR1 | VH CDR2 | VH CDR3 |
|---|---|---|---|
| Antibody | (SEQ ID NO:) | (SEQ ID NO:) | (SEQ ID NO:) |
| [Ag]AB-2 | SEQUENCE | SEQUENCE | SEQUENCE |
| cHv83-3a23.G1 | (SEQ ID NO: 43) | (SEQ ID NO: 44) | (SEQ ID NO: 45) |
| (L328C)k | SYVMH | GVSSSGDSTFYVDSVKG | GGITGAPLVFDI |
| [Ag]AB-3 | SEQUENCE | SEQUENCE | SEQUENCE |
| cHv83-1b15.G1 | (SEQ ID NO: 46) | (SEQ ID NO: 47) | (SEQ ID NO: 48) |
| (L328C)k | SHIMY | GISSNGLSSYYVDSVKG | GGRDRVPAVFDY |

TABLE 11
VL FR Amino Acid Sequences

| | VL FR1 | VL FR2 | VL FR3 | VL FR4 |
|---|---|---|---|---|
| Antibody | (SEQ ID NO:) | (SEQ ID NO:) | (SEQ ID NO:) | (SEQ ID NO:) |
| [Ag]AB-2 | SEQUENCE | SEQUENCE | SEQUENCE | SEQUENCE |
| cHv83-3a23.G1 | (SEQ ID NO: 49) | (SEQ ID | (SEQ ID NO: 51) | (SEQ ID |
| (L328C)k | DIQMTQSPSSLSASV | NO: 50) | GVPSRFSGSGSGTDF | NO: 52) |
| | GDRVTITC | WYQQKPGK | TLTISSLQPEDFATY | FGQGTR |
| | | APKVLIY | YC | LEIKR |
| [Ag]AB-3 | SEQUENCE | SEQUENCE | SEQUENCE | SEQUENCE |
| cHv83-1b15.G1 | (SEQ ID NO: 53) | (SEQ ID | (SEQ ID NO: 55) | (SEQ ID |
| (L328C)k | DIQMTQSPSSLSASI | NO: 54) | GVPSRFIGSGSGTDF | NO: 56) |
| | GDRVTITC | WYQQKPGK | TLTISSLQPEDFATY | FGQGTR |
| | | APKVLIY | YC | LEIKR |

TABLE 12
VH FR Amino Acid Sequences

| | | VH FR2 | | VH FR4 |
|---|---|---|---|---|
| | VH FR1 | (SEQ ID | VH FR3 | (SEQ ID |
| Antibody | (SEQ ID NO:) | NO:) | (SEQ ID NO:) | NO:) |
| [Ag]AB-2 | SEQUENCE | SEQUENCE | SEQUENCE | SEQUENCE |
| cHv83-3a23.G1 | (SEQ ID NO: 57) | (SEQ ID | (SEQ ID NO: 59) | (SEQ ID |
| (L328C)k | EVQLVESGGGLVQPG | NO: 58) | RFTISRDNSKNTLYL | NO: 60) |
| | GSLRLSCAASGFTFS | WVRQAPG | QMGSLRAEDMAVYYC | WGQGTM |
| | | KGLEYVS | AR | VTVSS |
| [Ag]AB-3 | SEQUENCE | SEQUENCE | SEQUENCE | SEQUENCE |
| cHv83-1b15.G1 | (SEQ ID NO: 61) | (SEQ ID | (SEQ ID NO: 63) | (SEQ ID |
| (L328C)k | EVQLVESGGGWVQPG | NO: 62) | RFTISRDNSKNLLYV | NO: 64) |
| | GSLRLSCAASGFTFS | WVRQAPG | HMGSLKPEDMAMYYC | WGQGTL |
| | | KGLEYVS | AR | VTVSS |

TABLE 13
VL Domain Amino Acid Sequences
Variable region (double underline), constant region (dotted underline)

| Antibody | VL (SEQ ID NO:) |
|---|---|
| [Ag] | SEQUENCE (SEQ ID NO: 67) |
| AB-2 | 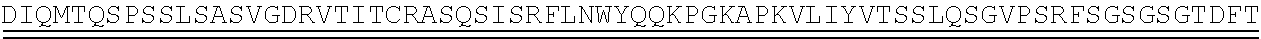 |
| cHv83- | 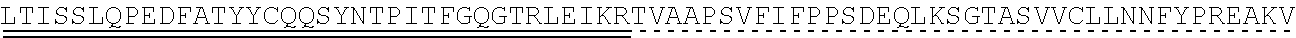 |
| 3a23.G1 | 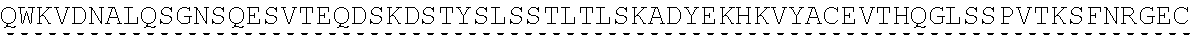 |
| (L328C)k | |
| [Ag] | SEQUENCE (SEQ ID NO: 68) |
| AB-3 | 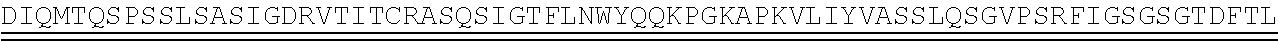 |
| cHv83- | 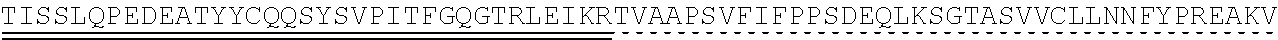 |
| 1B15.G1 | 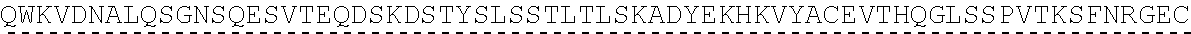 |
| (L328C)k | |

TABLE 14
VH Domain Amino Acid Sequences
Variable region (double underline), constant region (dotted underline)

| Antibody | VH (SEQ ID NO:) |
|---|---|
| [Ag] | SEQUENCE (SEQ ID NO: 67) |
| AB-2 | 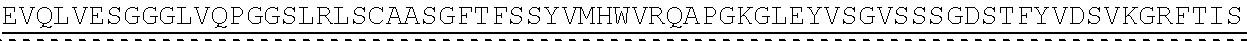 |
| cHv83- | 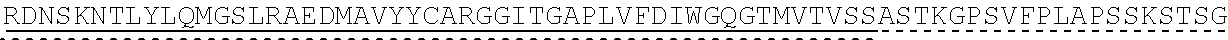 |
| 3a23.G1 | 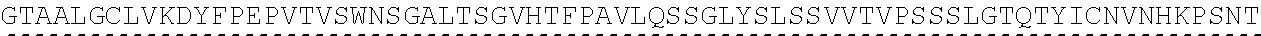 |
| (L328C)k | 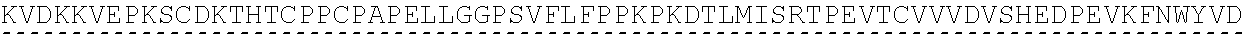 |
| | 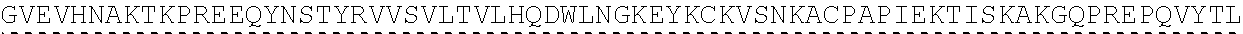 |
| | 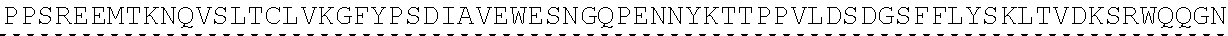 |
| | 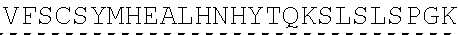 |
| [Ag] | SEQUENCE (SEQ ID NO: 68) |
| AB-3 | 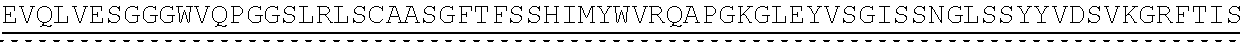 |
| cHv83- | 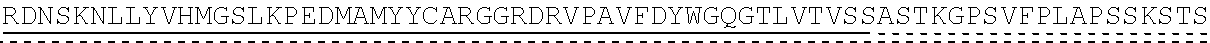 |
| 1b15.G1 | 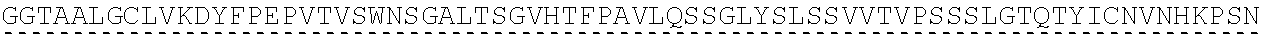 |
| (L328C)k | 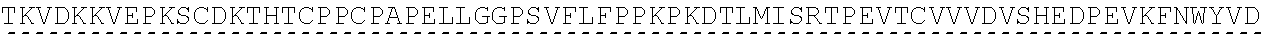 |
| | 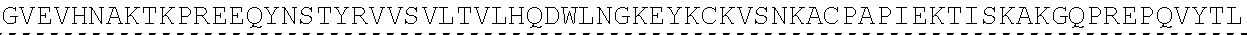 |
| | 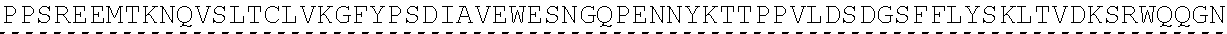 |
| | 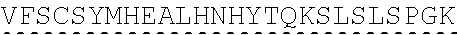 |

TABLE 15
VL Nucleic Acid Sequences

| Anti- | |
|---|---|
| body | Nucleotide sequences |
| [Ag] | SEQUENCE |
| AB-2 | (SEQ ID NO: 69) |
| cHv83- | GACATCCAGATGACCCAGTCTCCATCCTCCCTGTCTGCA |
| 3a23.G1 | TCTGTAGGAGACAGAGTCACCATCACTTGCCGGGCAAGT |
| (L328C) | CAGAGCATTAGCAGGTTTTTAAATTGGTATCAGCAGAAA |
| k | CCAGGGAAAGCCCCTAAGGTCCTGATCTATGTTACATCC |
| | AGTTTACAAAGTGGGGTCCCATCAAGGTTCAGTGGCAGT |
| | GGATCTGGGACAGATTTCACTCTCACCATCAGCAGTCTG |
| | CAACCTGAAGATTTTGCAACTTATTACTGTCAACAGAGT |
| | TACATACCCCTATCACCTTCGGCCAAGGGACACGACTGG |
| | AGATTAAACGGACTGTCGCTGCACCATCTGTCTTCATCT |
| | TCCCGCCATCTGATGAGCAGTTGAAATCTGGAACTGCCT |
| | CTGTTGTGTGCCTGCTGAATAACTTCTATCCCAGAGAGG |
| | CCAAAGTACAGTGGAAGGTGGATAACGCCCTCCAATCGG |
| | GTAACTCCCAGGAGAGTGTCACAGAGCAGGACAGCAAGG |
| | ACAGCACCTACAGCCTCAGCAGCACCCTGACGCTGAGCA |
| | AAGCAGACTACGAGAAACACAAAGTCTACGCCTGCGAAG |
| | TCACCCATCAGGGCCTGAGCTCGCCCGTCACAAAGAGCT |
| | TCAACAGGGGAGAGTGT |
| [Ag] | SEQUENCE |
| AB-3 | (SEQ ID NO: 70) |
| cHv83- | GACATCCAGATGACCCAGTCTCCATCCTCCCTGTCTGCA |
| 1b15.G1 | TCTATAGGAGACAGAGTCACCATCACTTGCCGGGCAAGT |
| (L328C) | CAGAGCATTGGCACCTTTTTAAATTGGTATCAACAAAAA |
| k | CCAGGGAAAGCCCCTAAGGTCCTGATCTATGTTGCATCC |
| | AGTTTGCAAAGTGGGGTCCCATCAAGGTTCATTGGCAGT |
| | GGATCTGGGACAGATTTCACTCTCACCATCAGCAGTCTG |
| | CAACCTGAAGATTTTGCAACTTACTACTGTCAACAGAGT |
| | TACAGTGTTCCGATCACCTTCGGCCAAGGGACACGACTG |
| | GAGATTAAACGGACTGTCGCTGCACCATCTGTCTTCATC |
| | TTCCCGCCATCTGATGAGCAGTTGAAATCTGGAACTGCC |
| | TCTGTTGTGTGCCTGCTGAATAACTTCTATCCCAGAGAG |
| | GCCAAAGTACAGTGGAAGGTGGATAACGCCCTCCAATCG |
| | GGTAACTCCCAGGAGAGTGTCACAGAGCAGGACAGCAAG |
| | GACAGCACCTACAGCCTCAGCAGCACCCTGACGCTGAGC |
| | AAAGCAGACTACGAGAAACACAAAGTCTACGCCTGCGAA |
| | GTCACCCATCAGGGCCTGAGCTCGCCCGTCACAAAGAGC |
| | TTCAACAGGGGAGAGTGT |

TABLE 16
VH Nucleic Acid Sequences

| Anti- | |
|---|---|
| body | Nucleotide sequences |
| [Ag] | SEQUENCE |
| AB-2 | (SEQ ID NO: 71) |
| cHv83- | GAGGTGCAGCTGGTGGAGTCTGGGGGAGGCTTGGTCCAGC |
| 3a23.G1 | CTGGGGGGTCCCTGAGACTCTCCTGTGCAGCCTCTGGATT |
| (L328C) | CACCTTCAGTAGTTATGTTATGCACTGGGTCCGCCAGGCT |
| k | CCAGGGAAGGGACTGGAATATGTTTCAGGTGTTAGTAGTA |
| | GTGGGGATAGCACATTTTATGTAGACTCTGTGAAGGGCAG |
| | ATTCACCATCTCCAGAGACAATTCCAAGAACACGCTTTAT |
| | CTTCAAATGGGCAGCCTGAGAGCTGAGGACATGGCTGTGT |
| | ATTACTGTGCGAGAGGGGGTATAACTGGAGCTCCACTGGT |
| | TTTTGATATCTGGGGCCAAGGGACAATGGTCACCGTCTCT |
| | TCAGCATCCACCAAGGGCCCATCGGTCTTCCCCCTGGCAC |
| | CCTCCTCCAAGAGCACCTCTGGGGGCACAGCGGCCCTGGG |
| | CTGCCTGGTCAAGGACTACTTCCCCGAACCGGTGACGGTG |
| | TCGTGGAACTCAGGCGCCCTGACCAGCGGCGTGCACACCT |
| | TCCCGGCTGTCCTACAGTCCTCAGGACTCTACTCCCTCAG |
| | CAGCGTGGTGACCGTGCCCTCCAGCAGCTTGGGCACCCAG |
| | ACCTACATCTGCAACGTGAATCACAAGCCCAGCAACACCA |
| | AGGTGGACAAGAAAGTTGAGCCCAAATCTTGTGACAAAAC |
| | TCACACATGCCCACCGTGCCCAGCACCTGAACTCCTGGGG |
| | GGACCGTCAGTCTTCCTCTTCCCCCCAAAACCCAAGGACA |
| | CCCTCATGATCTCCCGGACCCCTGAGGTCACATGCGTGGT |
| | GGTGGACGTGAGCCACGAAGACCCTGAGGTCAAGTTCAAC |
| | TGGTACGTGGACGGCGTGGAGGTGCATAATGCCAAGACAA |
| | AGCCGCGGGAGGAGCAGTACAACAGCACGTACCGTGTGGT |
| | CAGCGTCCTCACCGTCCTGCACCAGGACTGGCTGAATGGC |
| | AAGGAGTACAAGTGCAAGGTCTCCAACAAAGCCTGCCCAG |
| | CCCCCATCGAGAAAACCATCTCCAAAGCCAAAGGGCAGCC |
| | CCGAGAACCACAGGTGTACACCCTGCCCCCATCCCGGGAG |
| | GAGATGACCAAGAACCAGGTCAGCCTGACCTGCCTGGTCA |
| | AAGGCTTCTATCCCAGCGACATCGCCGTGGAGTGGGAGAG |
| | CAATGGGCAGCCGGAGAACAACTACAAGACCACGCCTCCC |
| | GTGCTGGACTCCGACGGCTCCTTCTTCCTCTATAGCAAGC |
| | TCACCGTGGACAAGAGCAGGTGGCAGCAGGGGAACGTCTT |
| | CTCATGCTCCGTGATGCATGAGGCTCTGCACAACCACTAC |
| | ACGCAGAAGAGCCTCTCCCTGTCTCCGGGTAAA |
| [Ag] | SEQUENCE |
| AB-3 | (SEQ ID NO: 72) |
| cHv83- | GAGGTGCAACTGGTGGAGTCTGGGGGAGGCTGGGTCCAGC |
| 1b15.G1 | CGGGGGGGTCCCTGAGACTCTCCTGTGCAGCCTCTGGATT |
| (L328C) | CACCTTCAGTAGTCATATTATGTACTGGGTCCGCCAGGCT |
| k | CCAGGGAAGGGACTGGAATATGTTTCGGGTATTAGCAGTA |
| | ATGGACTTAGCTCATATTATGTTGACTCTGTGAAGGGCAG |
| | ATTCACCATCTCCAGAGACAATTCCAAGAATTTACTGTAT |
| | GTTCATATGGGCAGCCTGAAACCTGAGGACATGGCTATGT |
| | ATTACTGTGCGAGAGGGGGCCGGGATAGAGTGCCAGCTGT |
| | CTTTGACTACTGGGGCCAGGGAACCCTGGTCACCGTCTCC |
| | TCCGCTTCCACCAAGGGCCCATCGGTCTTCCCCCTGGCAC |
| | CCTCCTCCAAGAGCACCTCTGGGGGCACAGCGGCCCTGGG |
| | CTGCCTGGTCAAGGACTACTTCCCCGAACCGGTGACGGTG |
| | TCGTGGAACTCAGGCGCCCTGACCAGCGGCGTGCACACCT |
| | TCCCGGCTGTCCTACAGTCCTCAGGACTCTACTCCCTCAG |
| | CAGCGTGGTGACCGTGCCCTCCAGCAGCTTGGGCACCCAG |
| | ACCTACATCTGCAACGTGAATCACAAGCCCAGCAACACCA |
| | AGGTGGACAAGAAAGTTGAGCCCAAATCTTGTGACAAAAC |
| | TCACACATGCCCACCGTGCCCAGCACCTGAACTCCTGGGG |
| | GGACCGTCAGTCTTCCTCTTCCCCCCAAAACCCAAGGACA |
| | CCCTCATGATCTCCCGGACCCCTGAGGTCACATGCGTGGT |
| | GGTGGACGTGAGCCACGAAGACCCTGAGGTCAAGTTCAAC |
| | TGGTACGTGGACGGCGTGGAGGTGCATAATGCCAAGACAA |
| | AGCCGCGGGAGGAGCAGTACAACAGCACGTACCGTGTGGT |
| | CAGCGTCCTCACCGTCCTGCACCAGGACTGGCTGAATGGC |
| | AAGGAGTACAAGTGCAAGGTCTCCAACAAAGCCTGCCCAG |
| | CCCCCATCGAGAAAACCATCTCCAAAGCCAAAGGGCAGCC |
| | CCGAGAACCACAGGTGTACACCCTGCCCCCATCCCGGGAG |
| | GAGATGACCAAGAACCAGGTCAGCCTGACCTGCCTGGTCA |
| | AAGGCTTCTATCCCAGCGACATCGCCGTGGAGTGGGAGAG |
| | CAATGGGCAGCCGGAGAACAACTACAAGACCACGCCTCCC |
| | GTGCTGGACTCCGACGGCTCCTTCTTCCTCTATAGCAAGC |
| | TCACCGTGGACAAGAGCAGGTGGCAGCAGGGGAACGTCTT |
| | CTCATGCTCCGTGATGCATGAGGCTCTGCACAACCACTAC |
| | ACGCAGAAGAGCCTCTCCCTGTCTCCGGGTAAA |

### EXAMPLES

### Example 1. Generation and Characterization of Anti-SLC34A2 Antibodies

To express SLC34A2 antibodies recombinantly in transfected Human embryonic kidney 293 cells, SLC34A2 antibody variable heavy and light chain sequences were cloned into plasmids constructs upstream of the human heavy chain IgG1 and human light chain Igκ constant regions respectively. The complete SLC34A2 antibody human heavy chain and light chain cassettes were cloned downstream of a promoter/enhancer in a cloning vector. A polyadenylation site was included downstream of the antibody coding sequence. The recombinant SLC34A2 antibody expressing constructs were transfected into 293 cells. The protein A purified SLC34A2 antibodies secreted from recombinant 293 cells were evaluated for binding to cell surface SLC34A2 by flow cytometry and by Biacore.

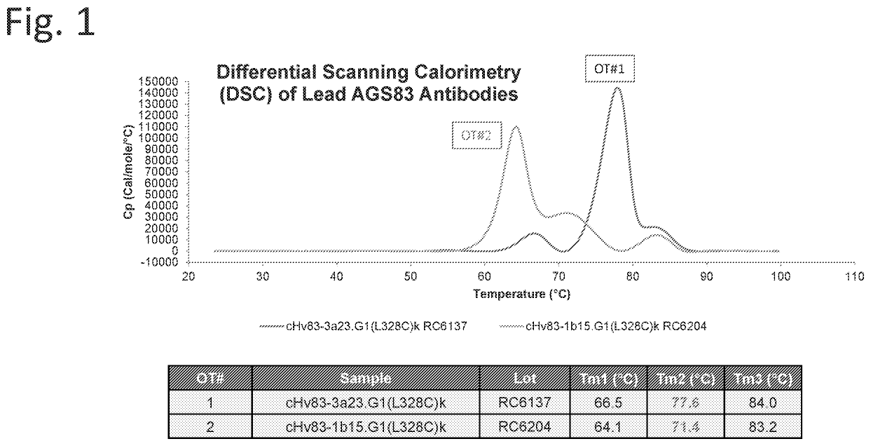

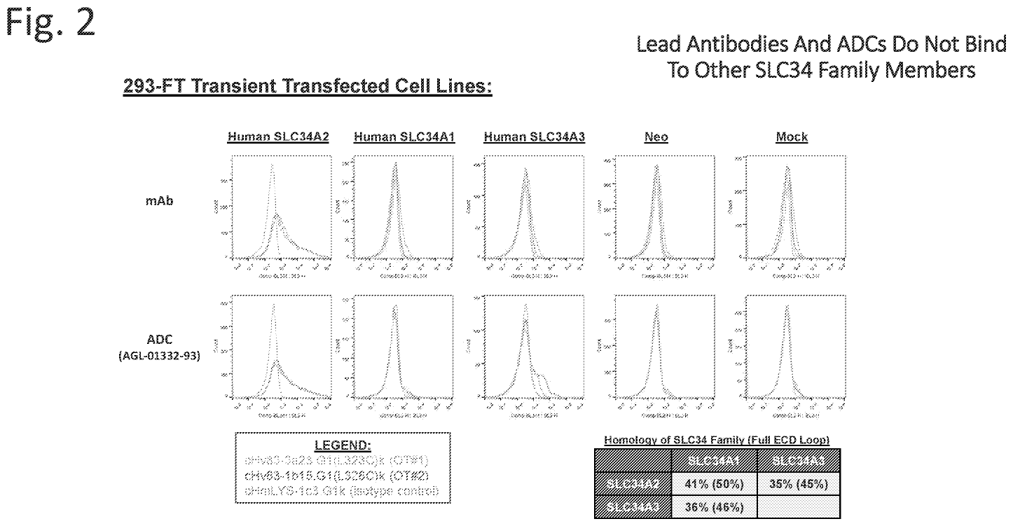

The purified antibodies were subsequently characterized by SDS-PAGE, SEC, CE-SDS, Differential Scanning Calorimetry (DSC), binding affinity determination, and paralog/homolog binding assessment. FIG. 1 showed DSC assays indicating the Tms of the two anti-SLC34A2 antibodies: Fab Tm of cHv83-3a23.G1(L328C)k was 77.6° C., and Fab Tm of cHv83-1b15.G1(L328C)k was 71.4° C., and OT #1 has superior in vitro thermal stability compared to OT #2. Table 1 shows the two anti-SLC34A2 antibodies bind to recombinant SLC34A2 with high affinity, and cross-reactive in human, cyno, and rats. FIG. 2 shows the anti-SLC34A2 antibodies and ADCs are specifically binding to SLC34A2, but not other SLC34 family proteins.

TABLE 1
Anti- SLC34A2 antibody binding affinities on recombinant SLC34A2

| | | | Rat 1E | | Rat 1E | | Rat 1E | |
|---|---|---|---|---|---|---|---|---|
| | OVCAR3 | | Human SLC34A2 | | Cyno SLC34A2 | | Murine SLC34A2 | |
| Antibody | Bmax | Kd (nM) | Bmax | Kd (nM) | Bmax | Kd (nM) | Bmax | Kd (nM) |
| cHv83- | 500207 | 2.278 | 68788 | 0.2543 | 92072 | 0.1703 | 11322 | 0.09035 |
| 3a23.G1(L328C)k | | | | | | | | |
| cHv83- | 538044 | 3.787 | 129552 | 1.027 | 59732 | 0.9671 | 15000 | 0.06311 |
| 1b15.G1k(L328C)k | | | | | | | | |
| cHmLYS-1c3.g1k | N.A. | N.A. | N.A. | N.A. | N.A. | N.A. | N.A. | N.A. |

### Example 2. Conjugation and Characterization of Anti-SLC34A2 Antibodies

Table 2 showed ADC Yield of the two antibodies. OT #1 conjugates to AGL-01332-93 consistently, while OT #2 displays atypical over-conjugation behavior, including high H2.

TABLE 2
ADC Yield of the two anti-SLC34A2 antibodies

| | AGL-01332-93 (BrAc) | | | AGL-01332-93Cl (Nal activated) | | | % |
|---|---|---|---|---|---|---|---|
| Antibody | n | avg DAR | Range | n | avg DAR | Range | Aggregation |
| OT1: cHv83- | 3 | 1.98 | 1.90-2.11 | 2 | 1.82 | 1.80-1.84 | 1.70 |
| 3a23.G1(L328C)k | | | | | | | |
| OT2: cHv83- | 1 | 2.43 | | 2 | 2.19 | 2.17-2.2 | 1.30 |
| 1b15.G1(L328C)k | | | | | | | |

Table 3 showed the binding affinity comparison between the anti-SLC34A2 antibodies and conjugated antibodies to SLC34A2, indicating that conjugation of the OT1 does not affect SLC34A2 binding.

TABLE 3
Anti- SLC34A2 antibodies and ADCs binding
affinity on recombinant SLC34A2

| | | Rat1E cells expressing SLC34A2 | | |
|---|---|---|---|---|
| | Epitope | Human | Cynomolgus | Rat |
| Antibody | bin | (nM) | (nM) | (nM) |
| cHv83-3a23.G1(L328C)k | 1 | 0.5526 | 0.1698 | 0.6888 |
| cHv83-3a23.G1(L328C)k- | 1 | 0.4108 | 0.2268 | 0.686 |
| AGL-01332-93 (OT#1) | | | | |
| cHv83-1b15.G1(L328C)k | 2a | 0.7214 | 0.6703 | 0.2456 |
| cHv83-1b15.G1(L328C)k- | 2a | 1.759 | 1.203 | 0.9127 |
| AGL-01332-93 (OT#2) | | | | |

The PK ECL followed a standard sandwich ELISA technique, with SLC34A2 protein being used as the capture protein. In brief, assay plates (Standard MSD plates) were coated with 50 μl of SLC34A2 at a concentration of 1 μg/ml and incubated overnight at 4° C. On day 2, the coating solution was washed with PBS/0.05% Tween20 wash buffer using the plate washer. 150 μL of blocking buffer was added and incubated at room temp for 1 hour followed by 3 washes with 300 μl/well of PBS/0.05% Tween20 using the plate washer. Serially diluted standard and serum study samples are pipetted into the wells. The 12-point standard curve in 1% Mouse; serum samples tested at 1:16200 dilution, ran in duplicates (50 μl/well). The controls were also added in duplicate. The plates were covered and incubated for 1 hour at room temp, then washed 3 times to remove the excess unbound substances and 50 μl/well of MSD Anti-Human IgG SulfoTag detection antibody for total protein and 50 μl/well of SG15.22 for ADC added in assay buffer. The plates were covered, incubated for 1 hour at room temp, washed 3 times. For total assay, 150 μl/well of MSD Read buffer (diluted to 2× with D.I. water) is added to the wells. The plates were then read on the MSD Meso Sector S600. For ADC assay, 50 μl/well of diluted MSD Streptavidine sulfo-tag was added. The plates were covered and incubated for 1 hour at room temp, then washed 3 times to remove the excess unbound detection antibody and 150 μl/well of MSD Read buffer (diluted to 2× with D.I. water) is added to the wells. The plates were then read on the MSD Meso Sector S600 and analyzed via MSD Discovery Workbench software.

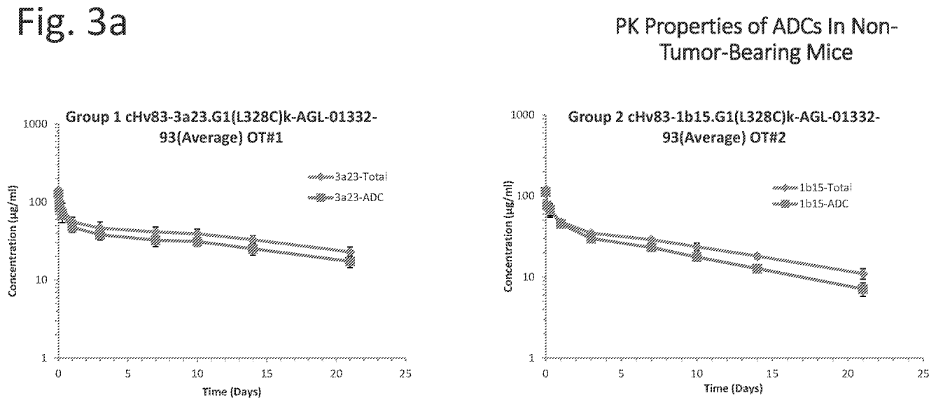

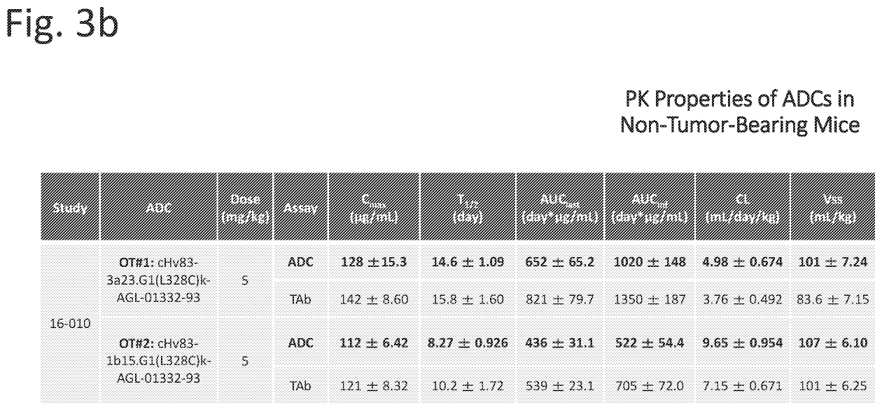

FIG. 3 showed stability of ADCs. Table 4. showed Pharmacokinetic Study of AGS-83 ADC in CB17/SCID non-tumor bearing mice. Minimal deconjugation and long half-lives were observed with both ADCs, and OT #1 exhibit better exposure and PK properties over OT #2.

TABLE 4
PK Properties of two anti- SLC34A2 ADC candidates.

| | Dose | | Cₘₐₓ | T_{1/2} | AUCₗₐₛₜ | AUC_{inf} | CL | Vss |
|---|---|---|---|---|---|---|---|---|
| ADC | (mg/kg) | Assay | (μg/mL) | (day) | (day*μg/mL) | (day*μg/mL) | (mL/day/kg) | (mL/kg) |
| OT#1: cHv83- | 5 | ADC | 128 ± 15.3 | 14.6 ± 1.09 | 672 ± 67.2 | 1020 ± 148 | 4.98 ± 0.674 | 101 ± 7.24 |
| 3a23.G1(L328C)k- | | TAb | 142 ± 8.60 | 15.8 ± 1.60 | 821 ± 79.7 | 1350 ± 187 | 3.76 ± 0.492 | 83.6 ± 7.15 |
| AGL-01332-93 | | | | | | | | |
| OT#2: cHv83- | 5 | ADC | 112 ± 6.42 | 8.27 ± 0.926 | 436 ± 31.1 | 522 ± 54.4 | 9.67 ± 0.954 | 107 ± 6.10 |
| 1b15.G1(L328C)k- | | TAb | 121 ± 8.32 | 10.2 ± 1.72 | 539 ± 23.1 | 705 ± 72.0 | 7.15 ± 0.671 | 101 ± 6.25 |
| AGL-01332-93 | | | | | | | | |

### Example 3. In Vitro Cytotoxicity of Conjugated Anti-SLC34A2 Antibodies

To assess in vitro cytotoxicity with SLC34A2 directed 01332-93 ADC, MDA Pca 2b cells were plated at 5000 cells/well in F-12 media (Gibco) with supplements in 96 well plates. After overnight culture at 37 degrees ADC were titrated into the cultures starting at 5 ug/mL. Cells were cultured with ADC for 6 days and cell viability was assessed by Cell Titre Glo (Promega) assay after 10′ incubation. Luminescence was determined on a Synergy plate reader (BioTek). % Survival vs. ADC concentration curves and EC50s were calculated with Graph Pad Prism software.

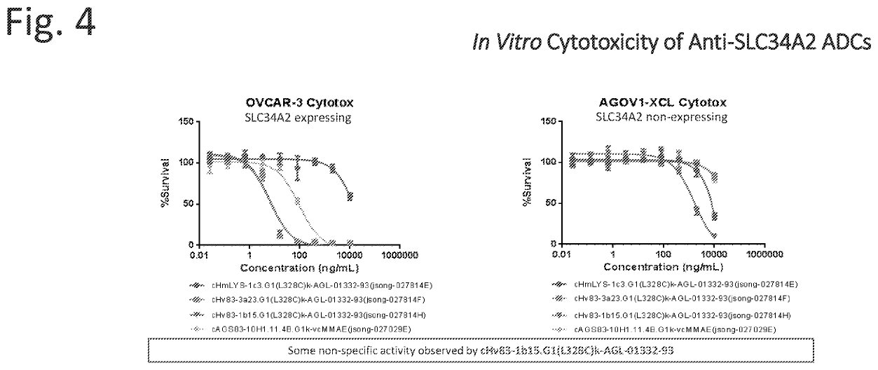

FIG. 4 indicated OT #1 demonstrates potent in vitro cytotoxicity and is more specific than OT #2.

### Example 4. In Vivo Efficacy of Conjugated Anti-SLC34A2 Antibodies in a OVCAR3 Tumor Mouse Model

Two to five pieces of OVCAR3 Ovarian tumors were implanted subcutaneously per female CB17/SCID or NSG mice 4-6 weeks of age. When the average tumor volumes reached approximately 200 mm³, mice were size matched and randomized into treatment and control groups before giving a single dose of AGS83 ADC intravenously at 2 mg/kg, and 5 mg/kg for each treatment group. Tumor size was determined by external caliper measurement twice a week.

A statistical analysis of the tumor volume data was performed using the Kruskal-Wallis test and the implementation of the Kruskal-Wallis test was carried out using the parametric ANOVA F-test on the ranks of the data. The percent tumor growth inhibition in each treated group versus a control group was calculated as [(Control-Control baseline)−(Treated-Treated baseline)]/(Control-Control baseline)×100%. The percent of tumor regression was defined as (Treated baseline-Treated)/Treated baseline×100%.

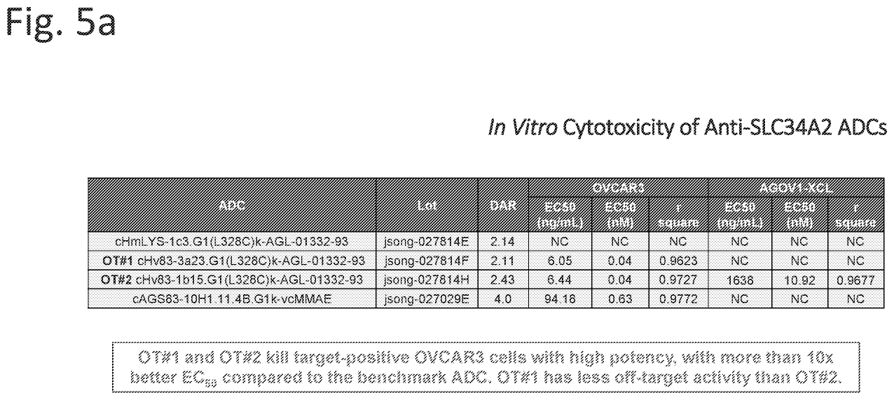

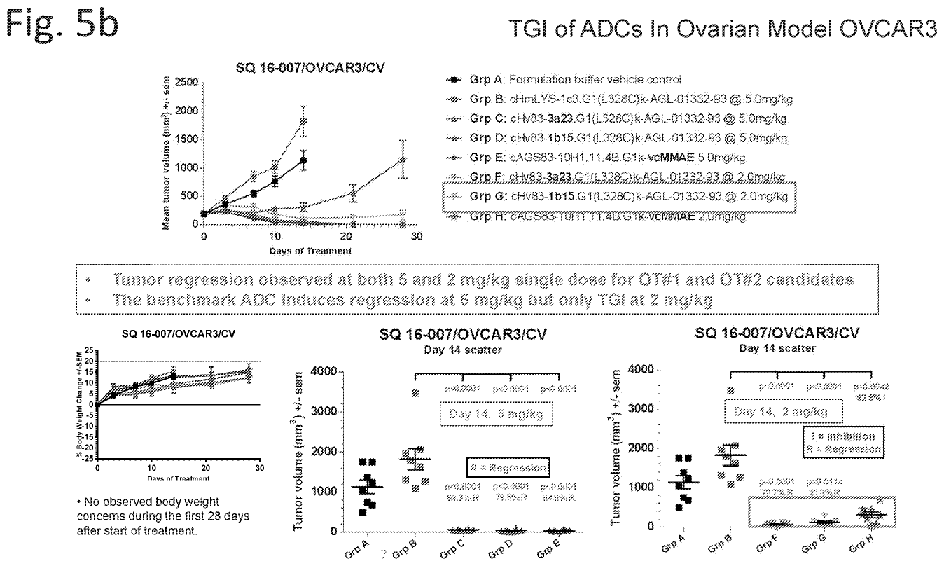

FIG. 5 showed OT #1 And OT #2 Exhibit Potent, Dose-Dependent TGI In Ovarian Model OVCAR3. In OVCAR3, both OT leads induced tumor regression at 5 mg/kg; both OT leads induced TGI that were statistically greater than the benchmark ADC at 2 mg/kg.

### Example 5. In Vivo Efficacy of Conjugated Anti-SLC34A2 Antibodies in an Ovarian PDX Model AG-OV37 Mouse Model

Two to five pieces of AG-OV37 Ovarian tumors were implanted subcutaneously per female CB17/SCID or NSG mice 4-6 weeks of age. When the average tumor volumes reached approximately 200 mm³, mice were size matched and randomized into treatment and control groups before giving a single dose of AGS83 ADC intravenously at 2 mg/kg, and 5 mg/kg for each treatment group. Tumor size was determined by external caliper measurement twice a week.

A statistical analysis of the tumor volume data was performed using the Kruskal-Wallis test and the implementation of the Kruskal-Wallis test was carried out using the parametric ANOVA F-test on the ranks of the data. The percent tumor growth inhibition in each treated group versus a control group was calculated as [(Control-Control baseline)−(Treated-Treated baseline)]/(Control-Control baseline)×100%. The percent of tumor regression was defined as (Treated baseline-Treated)/Treated baseline×100%.

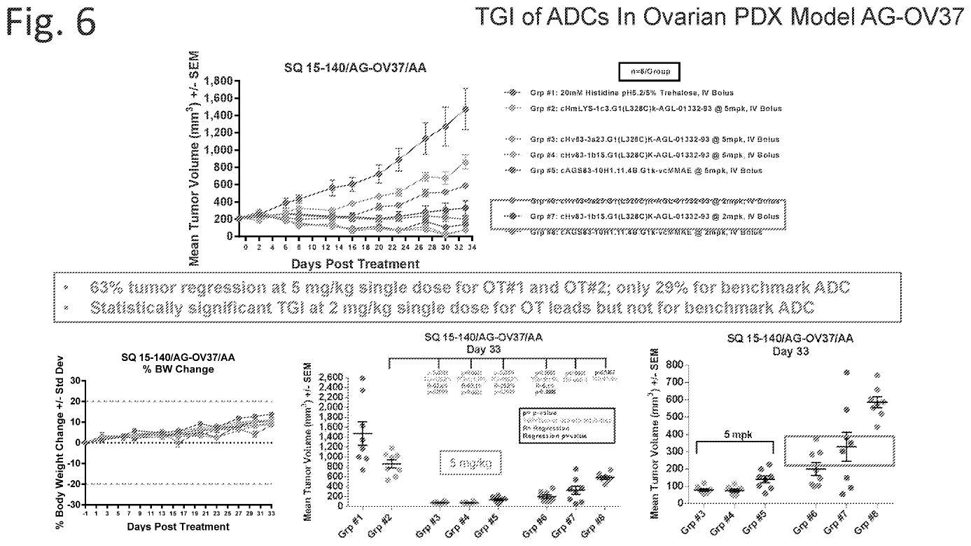

FIG. 6 showed OT #1 Exhibits More Potent TGI Than OT #2 And Benchmark ADC In Ovarian PDX Model AG-OV37. In AG-OV37, both OT leads induced statistically greater TGI than the benchmark ADC at both 2 and 5 mg/kg; OT #1 induced regression at 2 mg/kg but OT #2 and the benchmark ADC did not.

### Example 6. In Vivo Efficacy of Conjugated Anti-SLC34A2 Antibodies in a Human Lung Cancer NCI-H441 Mouse Model

Two to five pieces of NCI-H441 Human Lung cancer cell line were implanted subcutaneously per CB17/SCID mice 4-6 weeks of age. When the average tumor volumes reached approximately 200 mm³, mice were size matched and randomized into treatment and control groups before giving a single dose of AGS83 ADC intravenously at 5 mg/kg for each treatment group. Tumor size was determined by external caliper measurement twice a week.

A statistical analysis of the tumor volume data was performed using the Kruskal-Wallis test and the implementation of the Kruskal-Wallis test was carried out using the parametric ANOVA F-test on the ranks of the data. The percent tumor growth inhibition in each treated group versus a control group was calculated as [(Control-Control baseline)−(Treated-Treated baseline)]/(Control-Control baseline)×100/6. The percent of tumor regression was defined as (Treated baseline-Treated)/Treated baseline×100%.

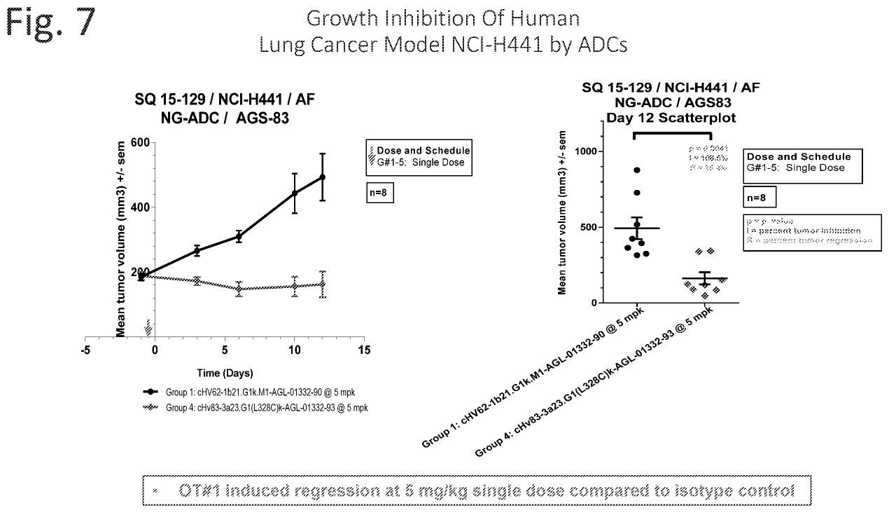

FIG. 7 showed OT #1 Inhibits Growth Of Human Lung Cancer Model NCI-H441. In NCI-H441, a single 5 mg/kg dose of OT #1 induced significant tumor growth inhibition and 16% regression compared to the isotype control.

### Example 7. In Vivo Efficacy of Conjugated Anti-SLC34A2 Antibodies in a Human Lung Cancer RERF-LC-Ad1 Mouse Model

Two to five pieces of RERF-LC-Ad1 Human Lung cancer cell line were implanted subcutaneously per CB17/SCID or NSG mice 4-6 weeks of age. When the average tumor volumes reached approximately 200 mm³, mice were size matched and randomized into treatment and control groups before giving a single dose of AGS83 ADC intravenously at 2.5 mg/kg or 5 mg/kg for each treatment group. Tumor size was determined by external caliper measurement twice a week.

A statistical analysis of the tumor volume data was performed using the Kruskal-Wallis test and the implementation of the Kruskal-Wallis test was carried out using the parametric ANOVA F-test on the ranks of the data. The percent tumor growth inhibition in each treated group versus a control group was calculated as [(Control-Control baseline)−(Treated-Treated baseline)]/(Control-Control baseline)×100%. The percent of tumor regression was defined as (Treated baseline-Treated)/Treated baseline×100%.

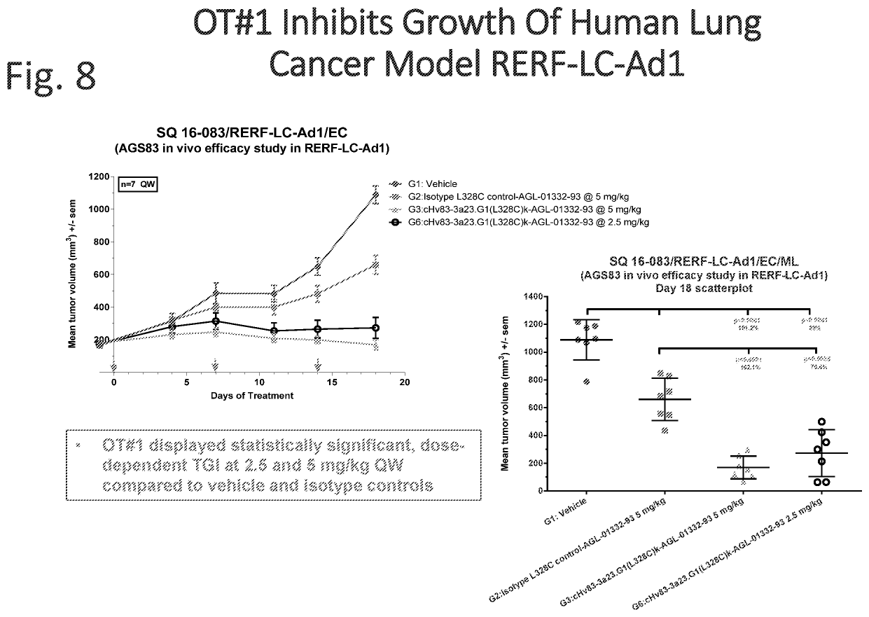

FIG. 8 showed OT #1 Inhibits Growth Of Human Lung Cancer Model RERF-LC-Ad1. In RERF-LC-Ad1, OT #1 induced dose-dependent, statistically significant tumor growth inhibition at 2.5 and 5 mg/kg weekly dosing.

### Example 8. Non-GLP Multiple-Dose Toxicity Study of Conjugated Anti-SLC34A2 Antibodies in Rats

Objective: Assess toxicity and toxicokinetics of cHv83-3a23.G1(L328C)k-AGL-01332-93 (OT #1)

Study Design

| | | Dose | | | | |
|---|---|---|---|---|---|---|
| | Group | | Level | Concentration | Volume | Number of Animals |
| Group | Color | Route | (mg/kg) | (mg/mL) | (mL/kg) ^{a} | Males |
| 1 | White | IV | 0 | 0 | 5 | 5^{b} + 6^{c} |
| 2 | Yellow | IV | 10 | 2 | 5 | 5^{b} + 6^{c} |
| 3 | Green | IV | 20 | 4 | 5 | 5^{b} + 6^{c} |
| 4 | Blue | IV | 40 | 8 | 5 | 5^{b} + 6^{c} |

Measures include In-life: clinical observations, body weight, food consumption, clinical pathology, urinalysis, BA/TK, and Post-mortem: gross pathology, organ weights, anatomic pathology.

Study design: 4-week study with no recovery period, with Dose levels: 0, 10, 20, 40 mg/kg

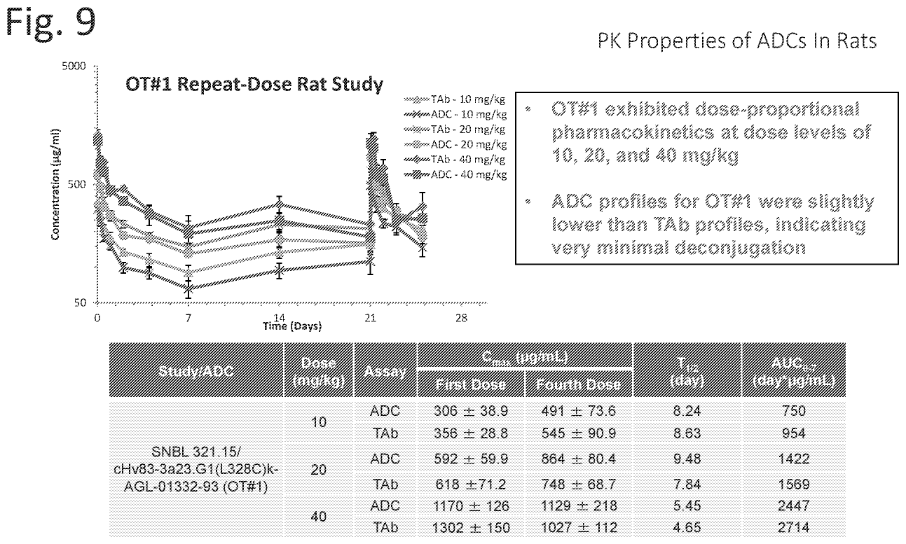

Results: Body weight loss (˜10%) on Day 29 and clinical observations of swelling (neck) at 40 mg/kg only. Effects on red cell mass, liver toxicity biomarkers, plasma proteins, and histopathology of bone marrow, kidney, liver, spleen considered potentially reversible (reversibility was not assessed). In general, findings at 40 mg/kg were of greater severity/incidence compared to 10 and 20 mg/kg. ADC localization correlated with areas of blood vasculature and not with histopathology findings. FIG. 9 showed OT #1 Exhibits favorable PK Properties in rats.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following.

What is claimed:

1. An isolated antibody or an antigen binding fragment thereof (AB) that specifically binds to mammalian SLC34A2, wherein the AB specifically binds human SLC34A2 and cynomolgus monkey SLC34A2, wherein the antibody or antigen-binding fragment thereof comprises a VH CDR1 amino acid sequence SYVMH (SEQ ID NO: 43); a VH CDR2 amino acid sequence GVSSSGDSTFYVDSVKG (SEQ ID NO: 44); a VH CDR3 amino acid sequence GGITGAPLVFDI (SEQ ID NO: 45); a VL CDR1 amino acid sequence RASQSISRFLN (SEQ ID NO: 37); a VL CDR2 amino acid sequence VTSSLQS (SEQ ID NO: 38); and a VL CDR3 amino acid sequence QQSYNTPIT (SEQ ID NO: 39).

2. The isolated antibody of claim 1, wherein the AB comprises a heavy chain variable region comprising the amino acid sequence of SEQ ID NO: 67, and a light chain variable region comprising an amino acid sequence selected from the group consisting of SEQ ID NOs: 67.

3. The isolated antibody of claim 1 or claim 2, wherein the antigen binding fragment thereof is selected from the group consisting of a Fab fragment, a F(ab′)₂ fragment, a scFv, and a scAb.

4. The isolated antibody of any one of claims 1 to 3, wherein the AB specifically binds human SLC34A2.

5. A conjugated antibody comprising the isolated antibody of any one of claims 1 to 4 conjugated to an agent.

6. The conjugated antibody of claim 5, wherein the agent is a toxin or fragment thereof.

7. The conjugated antibody of claim 6, wherein the agent is a microtubule inhibitor.

8. The conjugated antibody of claim 7, wherein the agent is selected from the group consisting of a dolastatin or a derivative thereof, an auristatin or a derivative thereof, a maytansinoid or a derivative thereof, a duocarmycin or a derivative thereof, a calicheamicin or a derivative thereof, and a pyrrolobenzodiazepine or a derivative thereof.

9. The conjugated antibody of claim 8, wherein the agent comprises a molecule having a structure of formula (I): 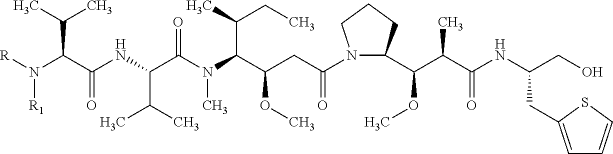
wherein R1 is a hydrogen or a C₁₋₆ alkyl group;
wherein R is selected from the group consisting of: a hydrogen, a C₁₋₆ alkyl, a linker, or a group X1-Y1-* wherein * is the point of attachment to the nitrogen; and
wherein Y1 is an oxycarbonyl group and X1 is a C₁₋₆alkyl group, a 9-fluorenylmethyl group, a benzyl group, or a tert-butyl group.

10. The conjugated antibody of any one of claims 5 to 9, wherein the agent is conjugated to the AB via a linker.

11. The conjugated antibody of claim 10, wherein the linker with a structure of formula (II): 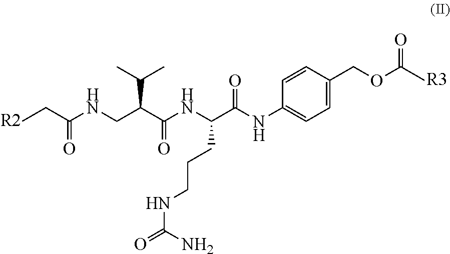
wherein R3 is an agent attached to formula (II) where the point of attachment is a nitrogen, sulfur, oxygen, or carbon atom; and
wherein R2 is an moiety attached to formula (II) wherein the point of attachment is selected from the group consisting of: a chlorine group, a iodine group, a bromine group, and a thiol group.

12. The conjugated antibody of claim 10, wherein the linker is a cleavable linker.

13. The conjugated antibody of claim 10, wherein the linker is a non-cleavable linker.

14. The conjugated antibody of claim 5, wherein the agent is a detectable moiety.

15. The conjugated antibody of claim 14, wherein the detectable moiety is a diagnostic agent.

16. The conjugated antibody of any one of claims 5 to 15, wherein the mammalian SLC34A2 is a human SLC34A2 and cynomolgus monkey SLC34A2.

17. The conjugated antibody of claim 5, wherein the agent is conjugated to the AB via a linker, and wherein the linker and the toxin have the structure of formula (III): 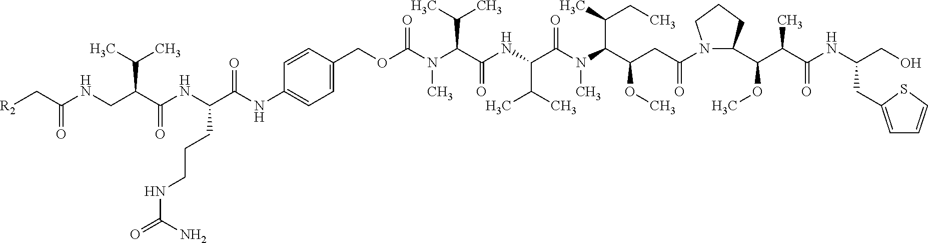
wherein R2 is a point of attachment to the AB.

18. The conjugated antibody of any one of claims 5 to 17, wherein the agent is conjugated to a thiol group on the AB.

19. The conjugated antibody of claim 18, wherein the thiol group is a cysteine side chain thiol group.

20. The conjugated antibody of claim 19, wherein the cysteine residue of the conjugated thiol group is at Kabat position 328 of the AB.

21. A conjugated antibody comprising:
an antibody or antigen binding fragment thereof (AB) that specifically binds to mammalian SLC34A2, wherein the AB comprises the VH CDR1 amino acid sequence SYVMH (SEQ ID NO: 43); a VH CDR2 amino acid sequence GVSSSGDSTFYVDSVKG (SEQ ID NO: 44), a VH CDR3 amino acid sequence GGITGAPLVFDI (SEQ ID NO: 45); a VL CDR1 amino acid sequence RASQSISRFLN (SEQ ID NO: 37); a VL CDR2 amino acid sequence VTSSLQS (SEQ ID NO: 38); and a VL CDR3 amino acid sequence QQSYNTPIT (SEQ ID NO: 39); and
an agent conjugated to the AB, wherein the agent comprises a molecule having a structure of formula (I): 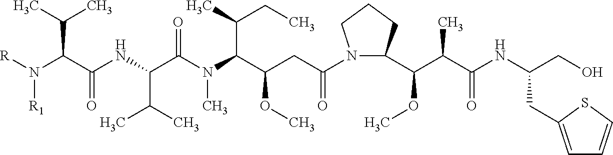
wherein R1 is a hydrogen or a C₁₋₆ alkyl group;
wherein R is selected from the group consisting of: a hydrogen, a C₁₋₆ alkyl, a linker, or a group X1-Y1-* wherein * is the point of attachment to the nitrogen; and
wherein Y1 is an oxycarbonyl group and X1 is a C₁₋₆ alkyl group, a 9-fluorenylmethyl group, a benzyl group, or a tert-butyl group.

22. The conjugated antibody of claim 21, wherein the AB comprises a heavy chain variable region comprising an amino acid sequence of SEQ ID NO: 67, and a light chain variable region comprising an amino acid sequence of SEQ ID NO: 67.

23. The conjugated antibody of claim 21 or claim 22, wherein the agent is conjugated to the AB via a linker.

24. The conjugated antibody of claim 23, wherein the linker with a structure of formula (II): 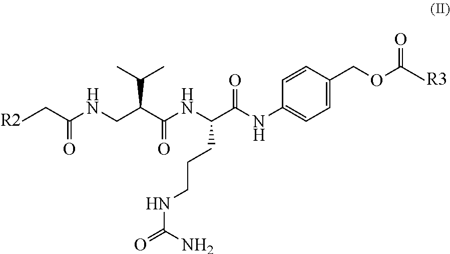
wherein R3 is point of attachment to the molecule of formula (I); and
wherein R2 is the point of attachment to the AB.

25. The conjugated antibody of claim 23, wherein the linker and the agent has a structure of formula (III): 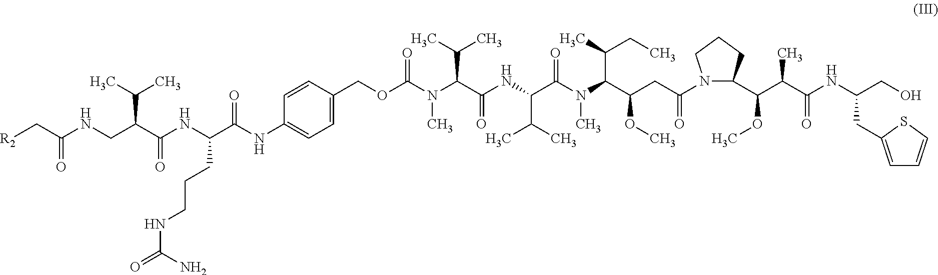
wherein R2 is the point of attachment is to the AB.

26. A pharmaceutical composition comprising the isolated antibody of any one of claims 1 to 4, or the conjugated antibody of any one of claims 5 to 25, and a carrier.

27. The pharmaceutical composition of claim 26 comprising an additional agent.

28. The pharmaceutical composition of claim 27, wherein the additional agent is a therapeutic agent.

29. An isolated nucleic acid molecule encoding the isolated antibody of any one of claims 1 to 4.

30. A vector comprising the isolated nucleic acid molecule of claim 29.

31. A method of producing an antibody by culturing a cell under conditions that lead to expression of the antibody, wherein the cell comprises the nucleic acid molecule of claim 29 or the vector of claim 30.

32. A method of manufacturing an antibody that binds SLC34A2, the method comprising: (a) culturing a cell comprising a nucleic acid construct that encodes the antibody of any one of claims 1 to 4 under conditions that lead to expression of the antibody; and (b) recovering the antibody.

33. A method of treating, alleviating a symptom of, or delaying the progression of a disorder or disease in which diseased cells express SLC34A2 comprising administering a therapeutically effective amount of the antibody of any one of claims 1 to 4, the conjugated antibody of any one of claims 5 to 25, or the pharmaceutical composition of claim 26 to a subject in need thereof.

34. A method of treating, alleviating a symptom of, or delaying the progression of a disorder or disease associated with cells expressing SLC34A2 comprising administering a therapeutically effective amount of the antibody of any one of claims 1 to 4, the conjugated antibody of any one of claims 5 to 25, or the pharmaceutical composition of claim 26 to a subject in need thereof.

35. The method of claim 33 or claim 34, wherein the disorder or disease associated with cells expressing SLC34A2 is cancer.

36. The method of claim 35, wherein the cancer is an ovarian cancer, a lung cancer, a non-small cell lung cancer (NSCLC), a small cell lung cancer (SCLC), a thyroid cancer, an endometrial cancer, a breast cancer, a Her2-negative breast cancer, a triple-negative breast cancer (TNBC), an estrogen receptor-positive breast cancer, or a kidney cancer.

37. A method of inhibiting or reducing the growth, proliferation, or metastasis of cells expressing mammalian SLC34A2 comprising administering a therapeutically effective amount of the antibody of any one of claims 1 to 4, the conjugated antibody of any one of claims 5 to 25, or the pharmaceutical composition of claim 26 to a subject in need thereof.

38. A method of inhibiting, blocking, or preventing the binding of a natural ligand or receptor to mammalian SLC34A2, comprising administering a therapeutically effective amount of the antibody of any one of claims 1 to 4, the conjugated antibody of any one of claims 5 to 25, or the pharmaceutical composition of claim 26 to a subject in need thereof.

39. The method of any one of claims 33 to 38, wherein the method comprises administering an additional agent.

40. The method of claim 39, wherein the additional agent is a therapeutic agent.


SEQUENCE LISTING (72 sequences)

SEQ ID NO: 1 - protein, 16 aa, Artificial Sequence
  Synthetic
RSSQSLLHSDGYNYLD

SEQ ID NO: 2 - protein, 7 aa, Artificial Sequence
  Synthetic
LGSNRAS

SEQ ID NO: 3 - protein, 9 aa, Artificial Sequence
  Synthetic
MQALQTPWT

SEQ ID NO: 4 - protein, 11 aa, Artificial Sequence
  Synthetic
RASQGISNWLA

SEQ ID NO: 5 - protein, 7 aa, Artificial Sequence
  Synthetic
AASSLQS

SEQ ID NO: 6 - protein, 9 aa, Artificial Sequence
  Synthetic
QQANSFPLT

SEQ ID NO: 7 - protein, 5 aa, Artificial Sequence
  Synthetic
SYDMH

SEQ ID NO: 8 - protein, 17 aa, Artificial Sequence
  Synthetic
VIWYDGSNKYYADSLKG

SEQ ID NO: 9 - protein, 14 aa, Artificial Sequence
  Synthetic
VIAARTFYYYGMDV

SEQ ID NO: 10 - protein, 5 aa, Artificial Sequence
  Synthetic
NYWMS

SEQ ID NO: 11 - protein, 17 aa, Artificial Sequence
  Synthetic
NIKKDGSEKFYVDSVKG

SEQ ID NO: 12 - protein, 8 aa, Artificial Sequence
  Synthetic
EIQLYLQH

SEQ ID NO: 13 - protein, 23 aa, Artificial Sequence
  Synthetic
DIVMTQSPLSLPVTPGEPASISC

SEQ ID NO: 14 - protein, 15 aa, Artificial Sequence
  Synthetic
WYLQKSGQSPQLLIY

SEQ ID NO: 15 - protein, 32 aa, Artificial Sequence
  Synthetic
GVPDRFSGSGSGTDFTLKISRVEAEDVGVYYC

SEQ ID NO: 16 - protein, 11 aa, Artificial Sequence
  Synthetic
FGQGTKVEIKR

SEQ ID NO: 17 - protein, 23 aa, Artificial Sequence
  Synthetic
DIQMTQSPSSVSASVGGRVTITC

SEQ ID NO: 18 - protein, 15 aa, Artificial Sequence
  Synthetic
WYQQKPGKAPKLLIY

SEQ ID NO: 19 - protein, 32 aa, Artificial Sequence
  Synthetic
GVPSRFSGSGSGTDFTLTISNLQPEDFASYYC

SEQ ID NO: 20 - protein, 11 aa, Artificial Sequence
  Synthetic
FGGGTKVEIKR

SEQ ID NO: 21 - protein, 30 aa, Artificial Sequence
  Synthetic
QVQLVESGGGVVQPGRSLRLSCAASGFTFS

SEQ ID NO: 22 - protein, 14 aa, Artificial Sequence
  Synthetic
WVRQAPGKGLEWVA

SEQ ID NO: 23 - protein, 32 aa, Artificial Sequence
  Synthetic
RFTISRDNSKNTLYLQMNSLRAEDTAVYYCAR

SEQ ID NO: 24 - protein, 11 aa, Artificial Sequence
  Synthetic
WGQGTTVTVSS

SEQ ID NO: 25 - protein, 30 aa, Artificial Sequence
  Synthetic
EVQLVESGGGLVQPGGSLRLSCAASGITFS

SEQ ID NO: 26 - protein, 14 aa, Artificial Sequence
  Synthetic
WVRQAPGKGLEWVA

SEQ ID NO: 27 - protein, 32 aa, Artificial Sequence
  Synthetic
RFTISRDNAKNSLYLQINSLRAEDTAMYYCAR

SEQ ID NO: 28 - protein, 11 aa, Artificial Sequence
  Synthetic
WGQGTLVTVSS

SEQ ID NO: 29 - protein, 219 aa, Artificial Sequence
  Synthetic
DIVMTQSPLSLPVTPGEPASISCRSSQSLLHSDGYNYLDWYLQKSGQSPQLLIYLGSNRASGVPDRFSGSGSGTDFTLKISRVEAEDVGVYYCMQALQTPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 214 aa, Artificial Sequence
  Synthetic
DIQMTQSPSSVSASVGGRVTITCRASQGISNWLAWYQQKPGKAPKLLIYAASSLQSGVPSRFSGSGSGTDFTLTISNLQPEDFASYYCQQANSFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 453 aa, Artificial Sequence
  Synthetic
QVQLVESGGGVVQPGRSLRLSCAASGFTFSSYDMHWVRQAPGKGLEWVAVIWYDGSNKYYADSLKGRFTISRDNSKNTLYLQMNSLRAEDTAVYYCARVIAARTFYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKACPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 447 aa, Artificial Sequence
  Synthetic
EVQLVESGGGLVQPGGSLRLSCAASGITFSNYWMSWVRQAPGKGLEWVANIKKDGSEKFYVDSVKGRFTISRDNAKNSLYLQINSLRAEDTAMYYCAREIQLYLQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKACPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - DNA, 657 bp, Artificial Sequence
  Synthetic
gatattgtgatgactcagtctccactctccctgcccgtcacccctggagagccggcctccatctcctgcaggtctagtcagagcctcctgcatagtgatggatacaactatttggattggtacctgcagaagtcagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactccgtggacgttcggccaagggaccaaggtggaaatcaaacggactgtcgctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 34 - DNA, 642 bp, Artificial Sequence
  Synthetic
gacatccagatgacccagtctccttcttccgtgtctgcatctgtaggaggcagagtcaccatcacttgtcgggcgagtcagggtattagcaactggttagcctggtatcagcagaaaccagggaaagcccctaaactcctgatctatgctgcatccagtttgcaaagtggggtcccatcaaggttcagcggcagtggatctgggacagatttcactctcaccatcagcaacctgcagcctgaagattttgcaagttactattgtcaacaggctaacagtttccccctcactttcggcggagggaccaaggtggagatcaaacggactgtcgctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 35 - DNA, 1359 bp, Artificial Sequence
  Synthetic
caggtgcagctggtggagtctgggggaggcgtggtccagcctgggaggtccctgagactctcctgtgcagcgtctggattcaccttcagtagctatgacatgcactgggtccgccaggctccaggcaaggggctggagtgggtggcagttatttggtatgatggaagtaataaatactatgcagactccttgaagggccgattcaccatctccagagacaattccaagaacacgctgtatctgcaaatgaacagcctcagagccgaggacacggctgtgtattactgtgcgagggttatagcagctcgtaccttctactactacggtatggacgtctggggccaagggaccacggtcaccgtctcctcagcatccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagcctgcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 36 - DNA, 1341 bp, Artificial Sequence
  Synthetic
gaggtgcagctggtggagtctgggggaggcttggtccagcctggggggtccctgagactctcctgtgcagcctctggaatcacctttagtaattattggatgagctgggtccgccaggctccagggaagggactggagtgggtggccaacataaagaaagatggaagtgagaaattctatgtggactctgtgaagggccgattcaccatctccagagacaacgccaagaactcactgtatctgcaaatcaacagcctgagagccgaggacacggctatgtattactgtgcgagagaaatacagctatacctgcagcactggggccagggcaccctggtcaccgtctcctcagcatccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagcctgcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 37 - protein, 11 aa, Artificial Sequence
  Synthetic
RASQSISRFLN

SEQ ID NO: 38 - protein, 7 aa, Artificial Sequence
  Synthetic
VTSSLQS

SEQ ID NO: 39 - protein, 9 aa, Artificial Sequence
  Synthetic
QQSYNTPIT

SEQ ID NO: 40 - protein, 11 aa, Artificial Sequence
  Synthetic
RASQSIGTFLN

SEQ ID NO: 41 - protein, 7 aa, Artificial Sequence
  Synthetic
VASSLQS

SEQ ID NO: 42 - protein, 9 aa, Artificial Sequence
  Synthetic
QQSYSVPIT

SEQ ID NO: 43 - protein, 5 aa, Artificial Sequence
  Synthetic
SYVMH

SEQ ID NO: 44 - protein, 17 aa, Artificial Sequence
  Synthetic
GVSSSGDSTFYVDSVKG

SEQ ID NO: 45 - protein, 12 aa, Artificial Sequence
  Synthetic
GGITGAPLVFDI

SEQ ID NO: 46 - protein, 5 aa, Artificial Sequence
  Synthetic
SHIMY

SEQ ID NO: 47 - protein, 17 aa, Artificial Sequence
  Synthetic
GISSNGLSSYYVDSVKG

SEQ ID NO: 48 - protein, 12 aa, Artificial Sequence
  Synthetic
GGRDRVPAVFDY

SEQ ID NO: 49 - protein, 23 aa, Artificial Sequence
  Synthetic
DIQMTQSPSSLSASVGDRVTITC

SEQ ID NO: 50 - protein, 15 aa, Artificial Sequence
  Synthetic
WYQQKPGKAPKVLIY

SEQ ID NO: 51 - protein, 32 aa, Artificial Sequence
  Synthetic
GVPSRFSGSGSGTDFTLTISSLQPEDFATYYC

SEQ ID NO: 52 - protein, 11 aa, Artificial Sequence
  Synthetic
FGQGTRLEIKR

SEQ ID NO: 53 - protein, 23 aa, Artificial Sequence
  Synthetic
DIQMTQSPSSLSASIGDRVTITC

SEQ ID NO: 54 - protein, 15 aa, Artificial Sequence
  Synthetic
WYQQKPGKAPKVLIY

SEQ ID NO: 55 - protein, 32 aa, Artificial Sequence
  Synthetic
GVPSRFIGSGSGTDFTLTISSLQPEDFATYYC

SEQ ID NO: 56 - protein, 11 aa, Artificial Sequence
  Synthetic
FGQGTRLEIKR

SEQ ID NO: 57 - protein, 30 aa, Artificial Sequence
  Synthetic
EVQLVESGGGLVQPGGSLRLSCAASGFTFS

SEQ ID NO: 58 - protein, 14 aa, Artificial Sequence
  Synthetic
WVRQAPGKGLEYVS

SEQ ID NO: 59 - protein, 32 aa, Artificial Sequence
  Synthetic
RFTISRDNSKNTLYLQMGSLRAEDMAVYYCAR

SEQ ID NO: 60 - protein, 11 aa, Artificial Sequence
  Synthetic
WGQGTMVTVSS

SEQ ID NO: 61 - protein, 30 aa, Artificial Sequence
  Synthetic
EVQLVESGGGWVQPGGSLRLSCAASGFTFS

SEQ ID NO: 62 - protein, 14 aa, Artificial Sequence
  Synthetic
WVRQAPGKGLEYVS

SEQ ID NO: 63 - protein, 32 aa, Artificial Sequence
  Synthetic
RFTISRDNSKNLLYVHMGSLKPEDMAMYYCAR

SEQ ID NO: 64 - protein, 11 aa, Artificial Sequence
  Synthetic
WGQGTLVTVSS

SEQ ID NO: 65 - protein, 214 aa, Artificial Sequence
  Synthetic
DIQMTQSPSSLSASVGDRVTITCRASQSISRFLNWYQQKPGKAPKVLIYVTSSLQSGVPSRFSGSGSGTDFTLTISSLQPEDFATYYCQQSYNTPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 214 aa, Artificial Sequence
  Synthetic
DIQMTQSPSSLSASIGDRVTITCRASQSIGTFLNWYQQKPGKAPKVLIYVASSLQSGVPSRFIGSGSGTDFTLTISSLQPEDFATYYCQQSYSVPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 451 aa, Artificial Sequence
  Synthetic
EVQLVESGGGLVQPGGSLRLSCAASGFTFSSYVMHWVRQAPGKGLEYVSGVSSSGDSTFYVDSVKGRFTISRDNSKNTLYLQMGSLRAEDMAVYYCARGGITGAPLVFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKACPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 451 aa, Artificial Sequence
  Synthetic
EVQLVESGGGWVQPGGSLRLSCAASGFTFSSHIMYWVRQAPGKGLEYVSGISSNGLSSYYVDSVKGRFTISRDNSKNLLYVHMGSLKPEDMAMYYCARGGRDRVPAVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKACPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - DNA, 642 bp, Artificial Sequence
  Synthetic
gacatccagatgacccagtctccatcctccctgtctgcatctgtaggagacagagtcaccatcacttgccgggcaagtcagagcattagcaggtttttaaattggtatcagcagaaaccagggaaagcccctaaggtcctgatctatgttacatccagtttacaaagtggggtcccatcaaggttcagtggcagtggatctgggacagatttcactctcaccatcagcagtctgcaacctgaagattttgcaacttattactgtcaacagagttacaatacccctatcaccttcggccaagggacacgactggagattaaacggactgtcgctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 70 - DNA, 642 bp, Artificial Sequence
  Synthetic
gacatccagatgacccagtctccatcctccctgtctgcatctataggagacagagtcaccatcacttgccgggcaagtcagagcattggcacctttttaaattggtatcaacaaaaaccagggaaagcccctaaggtcctgatctatgttgcatccagtttgcaaagtggggtcccatcaaggttcattggcagtggatctgggacagatttcactctcaccatcagcagtctgcaacctgaagattttgcaacttactactgtcaacagagttacagtgttccgatcaccttcggccaagggacacgactggagattaaacggactgtcgctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 71 - DNA, 1353 bp, Artificial Sequence
  Synthetic
gaggtgcagctggtggagtctgggggaggcttggtccagcctggggggtccctgagactctcctgtgcagcctctggattcaccttcagtagttatgttatgcactgggtccgccaggctccagggaagggactggaatatgtttcaggtgttagtagtagtggggatagcacattttatgtagactctgtgaagggcagattcaccatctccagagacaattccaagaacacgctttatcttcaaatgggcagcctgagagctgaggacatggctgtgtattactgtgcgagagggggtataactggagctccactggtttttgatatctggggccaagggacaatggtcaccgtctcttcagcatccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagcctgcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 72 - DNA, 1353 bp, Artificial Sequence
  Synthetic
gaggtgcaactggtggagtctgggggaggctgggtccagccgggggggtccctgagactctcctgtgcagcctctggattcaccttcagtagtcatattatgtactgggtccgccaggctccagggaagggactggaatatgtttcgggtattagcagtaatggacttagctcatattatgttgactctgtgaagggcagattcaccatctccagagacaattccaagaatttactgtatgttcatatgggcagcctgaaacctgaggacatggctatgtattactgtgcgagagggggccgggatagagtgccagctgtctttgactactggggccagggaaccctggtcaccgtctcctccgcttccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagcctgcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa